(19) United States
(12) Patent Application Publication
(10) Pub. No.: US 2019/0046647 A1
(43) Pub. Date: Feb. 14, 2019

(54) METHODS FOR TREATMENT OF CERVICAL INSUFFICIENCY

(71) Applicants: Trustees of Tufts College, Medford, MA (US); Tufts Medical Center, Boston, MA (US); Massachusetts Institute of Technology, Cambridge, MA (US)

(72) Inventors: Michael House, Lexington, MA (US); David L. Kaplan, Concord, MA (US); Errol Norwitz, Madison, CT (US); Simona Socrate, Cambridge, MA (US)

(21) Appl. No.: 15/914,065

(22) Filed: Mar. 7, 2018

Related U.S. Application Data
(63) Continuation of application No. 14/345,758, filed on Mar. 19, 2014, now abandoned.
(60) Provisional application No. 61/538,410, filed on Sep. 23, 2011.

(51) Int. Cl. A61K 47/42; A61L 27/36; A61K 47/20; A61K 47/10; A61K 35/36; A61L 27/22; A61L 27/52

(52) U.S. Cl. CPC A61K 47/42; A61L 27/3679; A61K 47/20; A61K 47/10; A61K 35/36; A61L 2430/34; A61L 2430/22; A61L 2400/06; A61L 27/52; A61L 27/3604; A61L 27/227

(57) ABSTRACT

Methods, compositions, devices and kits for increasing mechanical stiffness of an incompetent or a dilated biological tissue in a subject are provided herein. The methods described herein involve placing (e.g., injecting) a silk fibroin-based composition into at least a portion of an incompetent or dilated tissue of a subject. In some embodiments, the silk fibroin-based composition can further comprise at least two PEG components that will crosslink together upon placement (e.g., injection) into a subject in need thereof. In specific embodiments, the methods, compositions, devices and kits can be used to increase mechanical stiffness of a cervical tissue in a subject, for example, for treatment of cervical insufficiency.

### CROSS REFERENCE TO RELATED APPLICATIONS

This Application claims the benefit under 35 U.S.C. § 119(e) of U.S. Provisional Application No. 61/538,410 filed Sep. 23, 2011, the content of which is incorporated herein by reference in its entirety.

### GOVERNMENT SUPPORT

This invention was made with government support under grant Nos. P41 EB002520 and 5 K12 HD000849 25 both awarded by the National Institutes of Health. The government has certain rights in the invention.

### TECHNICAL FIELD OF THE DISCLOSURE

The inventions described herein relate to methods and compositions for increasing mechanical stiffness of a biological tissue in a subject. In specific embodiments, the methods and compositions can be used to increase mechanical stiffness of a cervical tissue in a subject, for example, for treatment of cervical insufficiency.

### BACKGROUND

A cervix normally undergoes a series of physical and biochemical changes during the course of pregnancy, which enhance the ease and safety of the birthing process for the mother and baby. Cervical softening occurs in a gradual manner throughout gestation. In the early stages of labor, the diameter of the proximal end of the cervical canal begins to increase at the internal orifice of the uterus (effacement of the internal os). During active labor, a period of regular uterine contractions, the cervix dilates allowing for the unobstructed passage of the fetus.

In addition to the physical and biochemical changes associated with normal labor, genetic or environmental factors, such as medical illness or infection, stress, malnutrition, chronic deprivation and/or certain chemicals or drugs can cause changes in the cervix. For example, it is well known that the in utero exposure of some women to diethylstilbestrol (DES) results in cervical abnormalities and in some cases gross anatomical changes, which leads to cervical insufficiency, where the cervix softens and dilates without apparent uterine contractions. Cervical insufficiency can also occur where there is a history of cervical injury, as in a previous traumatic delivery, or as a result of induced abortion if the cervix is forcibly dilated to large diameters. However, in many women with cervical insufficiency, there is no apparent etiology. Details of cervical insufficiency are discussed in Sonek, et al., Preterm Birth, Causes, Prevention and Management, Second Edition, McGraw-Hill. Inc., (1993), Chapter 5, which is incorporated by reference herein.

Cervical insufficiency (also known as “cervical incompetence”) arises when a cervix (the lower part of the uterus) is too weak to stay closed during a pregnancy. It is characterized as a condition in which the cervix fails to retain the conceptus during pregnancy. This can result in a preterm birth and possibly the loss of the baby because of previable birth. Cervical insufficiency has long been recognized as a potential cause of preterm delivery and recurrent mid trimester miscarriage. It is believed that cervical insufficiency is the cause of 20-25% of all second trimester losses in humans. Cervical insufficiency generally appears in the early part of the second trimester, but can be as late as the early third trimester in humans.

Cervical insufficiency is a clinical diagnosis. The diagnosis of cervical insufficiency is generally made in the setting of a preterm or previable birth in which uterine contractility is not prominent. The most important aspect of making the diagnosis is a careful review of the obstetric history. In addition to obstetric history, several findings have been associated with cervical insufficiency. It has been previously reported that increased internal cervical os diameter can be an indicator of cervical insufficiency (see Brook et al., J. Obstet. Gynecol. 88:640 (1981); Michaels et al., Am. J. Obstet. Gynecol. 154:537 (1986); Sarti et al., Radiology 130:417 (1979); and Vaalamo et al., Acta Obstet. Gynecol. Scan 62:19 (1983)). Internal os diameters ranging between 15 mm to 25 mm or wider have been observed in connection with cervical insufficiency. In addition, a short cervix (less than 25 mm) in the midtrimester can be indicative of cervical insufficiency. A clinical characteristic of cervical insufficiency can include preterm birth in the midtrimester at progressively earlier gestational ages in successive pregnancies.

Cervical insufficiency can be treated by cervical cerclage, which is a surgical technique that reinforces the cervical tissue by placing sutures in the cervical stroma to narrow the cervical canal and prevent it from opening before the pregnancy has gone to term. In humans, cervical cerclage can be done preventively at 12 to 14 weeks before the cervix thins out, or as an emergency measure after the cervix has thinned or dilated. Prior to cerclage placement, a speculum (an instrument with spoon-like paddles) is generally inserted into the pregnant woman's vagina to expose the cervix for the surgery.

The cerclage procedure can be done in different ways. For instance, the McDonald procedure is done with a permanent suture that is placed high on the cervix. The McDonald procedure can be performed in an elective setting (about 12-14 weeks) or in an emergency (after the cervix has shortened or dilated). In the McDonald procedure, the cervix is closed using four of five bites with a needle to create a purse string around the cervix. In another approach, a special tape can be tied around the cervix and stitched in place. In still another approach, a small incision can be made in the cervix. A special tape can then be tied through the cervix to close it. Other cerclage procedures can include the Shirodkar operation, the Hefner cerclage (also known as the Wurm procedure), the transabdominal cerclage by Benson and Durfee, and the Lash procedure.

Such vaginal approaches for cerclage placement are generally performed under spinal anesthesia. An exemplary product used for cerclage is the Ethicon product: Mersilene White Woven Polyester Fiber Ligature 5 mm on a M0-4 needle. The cerclage can be left in place until 37 weeks gestational age. At 37 weeks gestational age, the cerclage suture can be cut and removed. However, the suture has to be removed earlier than 37 weeks if preterm labor begins or preterm premature rupture of membranes occurs.

The primary pathophysiology of cervical insufficiency is weakened mechanical load bearing properties of the cervical tissue (see, e.g., Myers K M et al., 2010. J Biomech Eng. 132:021003; House M. et al. 2009 Semin Perinatol. 33: 300; and Myers K M et al., 2008. Acta Biomater. 4: 104), but cervical cerclage does not address this primary problem. Further, in some circumstances, cerclage therapy is not effective. Miscarriage or preterm birth can occur even though a cerclage is present. The natural history of cerclage failure can occur in several ways: (1) a cerclage can tear the tissue leading to cervical trauma; (2) a cerclage can become displaced and thus provides no load bearing support; (3) fetal membranes can protrude through the cervix even though a cerclage is present; (4) cervical insufficiency can occur in twins or triplets but cerclage is ineffective in these patient populations; (5) surgical placement of cerclage can be challenging, and such operative approach does not allow access to the upper part of the cervix, which is where failure occurs; and (6) intrauterine infection or utero-placental bleeding can occur in the presence of a cerclage. As such, there is still a strong need for novel methods that can enhance the mechanical load bearing properties of a cervical tissue, e.g., for treatment of cervical insufficiency.

### SUMMARY

Embodiments of various aspects described herein are based on, at least in part, an injectable silk fibroin-based composition for increasing a mechanical property of a biological tissue in a subject, e.g., a cervical tissue, for example, for treatment of cervical insufficiency.

Accordingly, one aspect provided herein is a method for increasing mechanical stiffness of a cervical tissue in a subject. The method comprises injecting a silk fibroin-based composition into at least a portion of a cervix of the subject, wherein the silk fibroin-based composition forms a gel upon injection into the cervix, thereby increasing the mechanical stiffness of the cervical tissue in the subject. In some embodiments, the injection can be performed, e.g., with a syringe and a needle, or a catheter.

In some embodiments, the methods described herein can further comprise exposing the cervix with a speculum prior to injection of the silk fibroin-based composition into the cervix of the subject.

In some embodiments, the silk fibroin-based composition can be injected into at least a portion of a stroma of the cervix.

In some embodiments, the silk fibroin-based composition can comprise silk fibroin at a concentration of about 5 wt % to about 30 wt %. In some embodiments, the silk fibroin can have a concentration of about 5 wt % to about 10 wt %.

In some embodiments, the silk fibroin-based composition can further comprise an active agent. Examples of the active agent can be selected from the group consisting of cells, proteins, peptides, nucleic acids, aptamers, antibodies or fragments or portions thereof, antigens, hormones, hormone antagonists, growth factors or recombinant growth factors and fragments and variants thereof, cell attachment mediators, enzymes, antibiotics, viruses, prodrugs, therapeutic agents, small molecules, and any combinations thereof. In particular embodiments, the active agent can be a population of cervical cells. In some embodiments, the active agent can be selected from a group consisting of thrombospondin 2, thrombin, fibrin, cyanoacrylate, glutaraldehyde, and any combinations thereof.

In various embodiments, the silk fibroin-based composition can further comprise an extracellular matrix protein, for example any art-recognized extracellular matrix protein present in the cervix. Examples of the extracellular matrix protein can include, but are not limited to, collagen, proteoglycans, hyaluronan, elastin, and any combinations thereof. In one embodiment, the silk fibroin-based composition can further comprise collagen.

Various approaches can be utilized to cause the silk fibroin-based composition to form a gel upon injection. For example, in some embodiments, the silk fibroin-based composition can further comprise at least two functionally-activated PEG components capable of reacting with one another to form a crosslinked matrix, and the silk fibroin capable of forming beta-sheets to further stabilize the crosslinked matrix. In such embodiments, each PEG component can be a four-armed PEG. In some embodiments, one of the PEG components can be functionally activated with a maleimidyl group. In some embodiments, one of the PEG components can be functionally activated with a thiol group. In certain embodiments, one of the PEG components can be functionally activated with a maleimidyl group and another one of the PEG components can be functionally activated with a thiol group.

In some embodiments where the silk fibroin-based composition comprising PEG components, the method described herein can further comprise mixing the PEG components and silk fibroin.

In alternative embodiments, the silk fibroin-based composition can form a gel in the cervix by exposing the silk fibroin-based composition, upon injection into the cervix, to a treatment comprising ultrasound for a sufficient period of time to initiate gelation.

In other embodiments, the silk fibroin-based composition can form a gel in the cervix by exposing the silk fibroin-based composition, upon injection into the cervix, to a treatment comprising an electric field for a sufficient period of time to initiate gelation.

The mechanical properties of the gel formed in the cervix can be modulated by altering the amount of beta-sheets formed by the silk fibroin. Methods for increasing the amount of beta-sheets in the silk fibroin-based gel are well known in the art, including, but not limited to, an alcohol treatment or a water-annealing treatment. Accordingly, in some embodiments, the silk fibroin-based composition can be exposed, upon injection into the cervix, to an alcohol treatment or a water-annealing treatment.

The method provided herein can be used to treat any subject in need thereof. Without wishing to be bound, in some embodiments, the subject in need thereof can be at risk of, or diagnosed with cervical insufficiency. In some embodiments, the subject can have a history of preterm birth. In some embodiments, the subject can have a cervical length of less than 2.5 cm. In some embodiments, the subject can be indicated to a treatment comprising a cervical cerclage. In some embodiments, the subject can be at risk of, or diagnosed with a multiple gestation.

In some embodiments, the method described herein can be used in conjunction with a treatment comprising a cervical cerclage and/or progesterone therapy.

Kits for treatment of an incompetent cervix or cervical insufficiency are also provided herein. In some embodiments, a kit comprises a delivery device containing a silk fibroin-based composition that forms a gel upon injection into a cervix, and a speculum. In some embodiments, the kit can further comprise an active agent described herein. The active agent can be provided in a separate container or mixed with the silk fibroin-based composition contained in the delivery device.

### BRIEF DESCRIPTION OF THE DRAWINGS

FIG. 1 is a set of images showing one embodiment of a silk fibroin-based composition that comprises silk fibroin and polyethylene glycol (PEG) contained in a double-barreled syringe with a mixing tip, and the silk fibroin-based gels after 2-step gelation under indicated conditions. Solution A comprised 10 wt % silk fibroin and 10 wt % PEG thiol, while solution B comprised 10 wt % silk fibroin and 10 wt % PEG maleimide. After the initial gelation of the silk fibroin-based composition due to crosslinking between the two PEG components (as shown in Phase 1: mixing of the two solutions generally results in a chemically crosslinked gel due to the reaction between the thiol and maleimide functional groups), the phase I gel was further subjected to various indicated treatments (e.g., methanol, MeOH; ethanol, EtOH; water, H2O; phosphate buffered saline, PBS) as shown in Phase 2 (“silk fibroin-PEG gel”). Further stabilization arises due to beta sheet formation in silk, which occurs when the silk-PEG is exposed to methanol or ethanol (white color) but not water or PBS.

FIGS. 2A-2C are images showing gross morphology and histology of a cervical biopsy before and after injections of the silk fibroin-based compositions in accordance with one or more embodiments described herein. FIG. 2A is a schematic diagram of a cervix with an indicated biopsy site. FIG. 2B is a set of images showing gross morphology of a cervical biopsy before and after injections of the silk fibroin-based composition indicated in FIG. 1. After injection, the cervical tissue appears larger due to the increase in mass. The silk-PEG appears as a translucent, white material on the tissue. FIG. 2C is a set of hematoxylin and eosin (H&E) histology images of cervical tissues after injection showing integration of the silk-PEG biomaterial into the tissue.

FIG. 3 is a graph showing increase in cervical tissue weights after injection of the silk fibroin-based composition indicated in FIG. 1. The mean increase in tissue wet weight after injection was about 17% with a standard deviation of about 6.7%.

FIG. 4 is a set of images showing gross morphology of a silk fibroin-PEG gel (left panel) and a cervical tissue obtained from a hysterectomy specimen (right panel).

FIGS. 5A-5C show an exemplary mechanical testing set-up and results of silk fibroin-PEG gels or cervical tissues injected with silk fibroin-based composition comprising PEG. FIG. 5A is an image of the experimental set-up for a uniaxial compression test. FIG. 5B is a stress-strain graph showing the mechanical performance of different silk fibroin-PEG gels subjected to uniaxial compression. In some embodiments, the silk fibroin-PEG gels comprised 5 wt % silk fibroin and either 10 wt % or 5 wt % of total PEG. The stress-strain graph indicates that higher percents of PEG and/or post-treatment with alcohol such as ethanol (EtOH) yield a stiffer gel (indicated by a higher elastic modulus E). FIG. 5C is a stress-strain graph showing the mechanical performance of a cervical tissue injected with a silk fibroin-based composition comprising 10 wt % PEG, when the injected tissue was subjected to uniaxial compression. The stress-strain graph indicates that the cervical tissue injected with one embodiment of the silk fibroin-based composition described herein is stiffer than the untreated one (e.g., native cervical tissue).

FIGS. 6A-6B show experimental results of the swelling properties of the cervical tissue, injected with one or more embodiments of the silk fibroin-based composition described herein, in phosphate buffered saline (PBS). FIG. 6A is an image showing gross morphology of swollen cervical tissues in PBS before the tissues were subjected to any injection of the silk fibroin-based composition. FIG. 6B shows the quantitative results of the swelling behavior of the cervical tissues injected with silk fibroin-based compositions comprising at least about 10 wt % silk fibroin. When the cervical tissue was injected with one or more embodiments of the silk fibroin-based composition comprising PEG, they demonstrated in PBS comparable swelling behavior as the native cervical tissue. The swelling property was determined by weight (e.g., percent in mass, in mg). Swelling was significantly increased (p<0.01) in the tissue injected with PEG alone.

FIGS. 7A-7D show experimental results of the mechanical characterization of a cervical tissue injected with one or more embodiments of a silk fibroin-based composition described herein. FIG. 7A is a picture of an exemplary setup for indentation testing with the cervical tissue specimen placed in PBS. Indentation was performed in the same spot before and after tissue injection. FIG. 7B is a line graph showing that a sample load-unload cycle on a cervical tissue specimen shows a consistent response between testing cycles (n=3 cycles). FIG. 7C is a line graph showing that the peak force needed to compress the cervical tissue by 20% increased significantly after injection of the silk/PEG biomaterial. FIG. 7D is a correlation graph showing a positive dose-response. As more silk-PEG biomaterial was injected, the peak force ratio significantly increased (p=0.002).

FIG. 8 is a set of fluorescent images showing cytocompatibility of cervical fibroblasts cultures on cervical tissues injected with silk-PEG biomaterial. Cervical fibroblasts are viable when cultured for 48 hours on cervical tissue injected with silk/PEG biomaterial. Viable cervical fibroblasts display a rounded morphology on the silk/PEG biomaterial and spindle shaped morphology on cervical tissue (inset). On the silk/PEG biomaterial alone (control), the cervical fibroblasts have a rounded morphology.

### DETAILED DESCRIPTION OF THE INVENTION

Various aspects and embodiments described herein generally relates to methods, compositions, devices and kits for increasing mechanical stiffness of a biological tissue in a subject. In accordance with various aspects described herein, a protein-based composition (e.g., silk fibroin-based composition) can be injected into a biological tissue that is determined to need a treatment for increasing its mechanical stiffness. For example, a protein-based composition (e.g., silk fibroin-based composition) can be injected into a weakened cervix of a pregnant subject to provide enhanced load-bearing support, which could otherwise lead to miscarriage or preterm birth if no treatment is administered. Thus, in specific embodiments, methods, compositions, devices and kits for increasing mechanical stiffness of a cervical tissue in a subject are provided herein. In these embodiments, a protein-based composition (e.g., silk fibroin-based composition) can be injected into a cervical tissue of a subject in need thereof. Once in the cervical tissue, the protein-based composition (e.g., silk fibroin-based composition) can form a gel, which can contribute to stiffening of the cervical tissue. The stiffness of the protein-based gel (e.g., silk fibroin-based gel) can be adjusted for, e.g., matching or exceeding the stiffness of a cervical native tissue, thereby providing load bearing support. Thus, the methods, compositions and kits described herein can be used to treat a subject in need of load-bearing support for her cervical tissue, for example, for treatment of cervical insufficiency.

### Methods for Increasing Mechanical Stiffness of a Cervical Tissue in a Subject

One aspect provided herein is a method for increasing mechanical stiffness of a cervical tissue in a subject. The method comprises placing a silk fibroin-based composition into at least a portion of a cervix of a subject, wherein the silk fibroin-based composition forms a gel upon placement into the cervix, thereby increasing the mechanical stiffness of the cervical tissue in the subject.

As used herein, the term “cervical tissue” refers to a tissue associated with a cervix, which is generally the lower part of a uterus. The cervix is a substantially cylindrical anatomical structure and comprises three tissue layers, namely, fascia, stroma and mucosa layers of the cervix, surrounding an inner canal (see e.g., Myers et al., 2010 Journal of Biomechanical Engineering 132(2): 021003). In some embodiments, the term “cervical tissue” can encompass any tissue near the internal orifice of the uterus (or internal orifice of the cervix uteri or internal os) and/or the external orifice of the uterus (or external orifice of the cervix uteri or external os). In some embodiments, the term “cervical tissue” can comprise at least a portion of a uterus muscle, e.g., lower and/or upper part of the uterus. In some embodiments, the term “cervical tissue” can comprise at least a portion of the vaginal tissue. in some embodiments, the term “cervical tissue” can comprise at least a portion of a uterine wall. In one embodiment, the cervical tissue can comprise a tissue immediately surrounding the cervix uteri.

The term “placing” as used herein refers to any means of delivering, introducing or administering the silk fibroin-based composition into at least a portion of a cervix or a cervical tissue in a subject. Without wishing to be bound, an exemplary method of placing a silk fibroin-based composition into a cervical tissue includes injection. Accordingly, in certain embodiments, the method described herein can comprise injecting a silk-fibroin based composition to at least a portion of a cervix of a subject, wherein the silk fibroin-based composition forms a gel upon injection into the cervix, thereby increasing the mechanical stiffness of the cervical tissue in the subject. Injection can be performed by any methods known in the art, e.g., via a syringe with a needle, a cannula, and/or a catheter. In some embodiments, the injection can be performed through an anatomical opening, e.g., a vagina. In some embodiments, the injection can be performed through an incision on a skin (e.g., abdominal skin), followed by insertion of a needle, a cannula, and/or tubing, e.g., a catheter.

In some embodiments, the silk fibroin-based composition can be injected into at least a portion of a stroma of the cervix. The stroma of the cervix can be characterized by three zones of collagen: the innermost and outmost rings of stroma typically contain collagen fibers preferentially aligned in the longitudinal direction, and the middle layer contains collagen fibers preferentially aligned in the circumferential direction (See, e.g., Aspden R. M. 1988. Coll. Relat. Res. 8: 103). In such embodiments, the silk fibroin-based composition can be injected into at least one layer of the stroma of the cervix.

The methods described herein can comprise one or more injections of the silk fibroin-based composition into at least a portion of a cervix. In some embodiments, the method described herein can comprise one injection into at least a portion of a cervix. In other embodiments, the method described herein can comprise a plurality of injections, for example, including at least 2 injections, at least 3 injections, at least 4 injections, at least 5 injections, at least 6 injections, at least 7 injections, at least 8 injections or more, of the silk fibroin-based composition into at least a portion of a cervix. The silk fibroin-based composition can be injected circumferentially around and/or longitudinally along a cervix. In some embodiments, the location of the injections can be the same as where the needle would enter and/or exit if a cerclage was performed.

In some embodiments, after the silk fibroin-based composition is placed into at least a portion of a cervix for a period of time (e.g., a period of about 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, or more), additional silk fibroin-based composition (e.g., the same or a different composition) can be placed into the cervix if needed.

Each injection volume can vary with a number of factors including, but not limited to, mechanical stiffness of the cervix to be treated, the silk fibroin-based composition and concentrations of the components thereof, and/or desirable mechanical stiffness to be achieved in the cervix. In some embodiments, the injection volume can range from about 0.01 mL to about 10 mL, from about 0.1 mL to about 5 mL, or from about 0.5 mL to about 4 mL.

In some embodiments, the methods described herein can further comprise exposing the cervix prior to injection of the silk fibroin-based composition into the cervix of the subject. The cervix can be exposed by, for example, stretching the vagina open, e.g., with a speculum, or any other methods known to a skilled practitioner.

Upon injection into the cervix, the silk fibroin-based composition forms a gel, thereby increasing the mechanical stiffness of the cervical tissue in the subject. For example, the gelation of the silk fibroin-based composition can increase the mechanical stiffness of the cervical tissue by at least about 30%, at least about 40%, at least about 50%, at least about 60%, at least about 70%, at least about 80%, at least about 90%, at least about 100%, at least about 200%, at least about 300%, at least about 400%, at least about 500% or more, as compared to that of a cervical tissue in the absence of the silk fibroin-based composition.

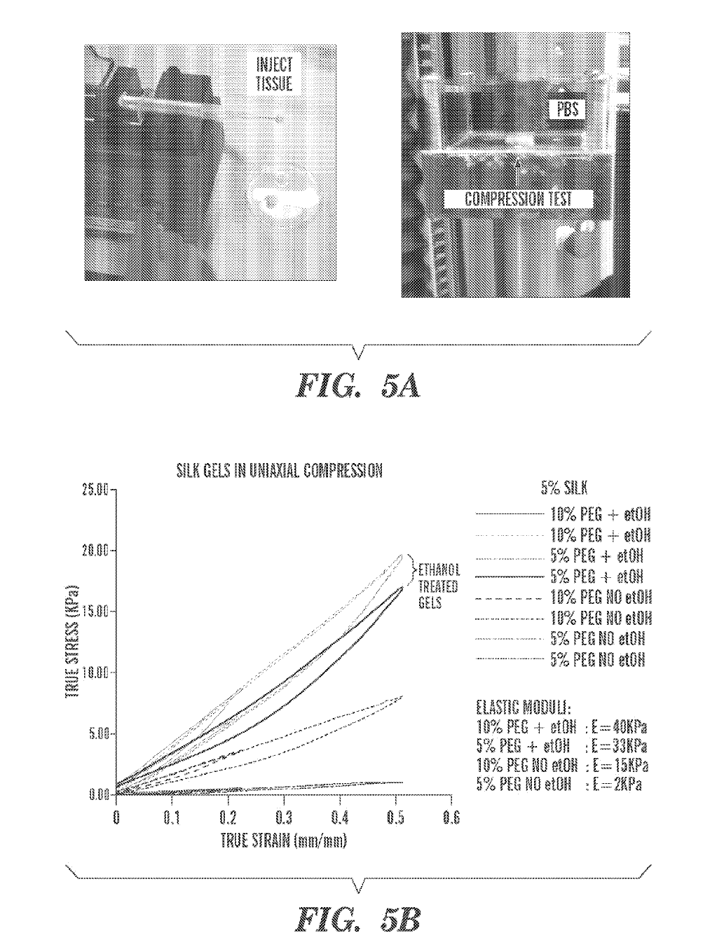

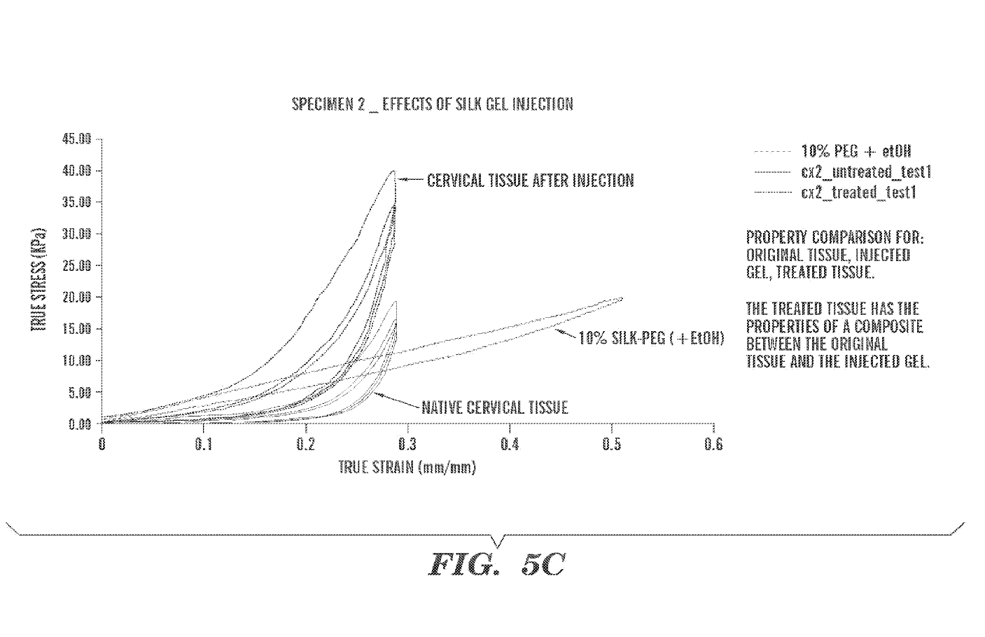

By the term “mechanical stiffness” is generally meant a property of a material characterized by its load-bearing strength, for example, an ability of the material to maintain its shape and size, and/or resist to deformation upon application of a pressure. Mechanical stiffness can encompass tensile stiffness and/or compressive stiffness. With respect to a cervical tissue, the mechanical stiffness of a cervical tissue can refer to a load-bearing strength of the cervical tissue. For example, the mechanical stiffness of a cervical tissue can be determined by measuring the level of mechanical stress required to produce a certain compressive strain, e.g., of about 5%, about 10%, about 15%, about 20%, about 25%, about 30%, about 40%, about 50% or more in the cervical tissue (see, e.g., FIGS. 5A-5C). In one embodiment, the mechanical stiffness of a cervical tissue can be determined by measuring the level of mechanical stress required to produce a uniaxial compressive strain of about 25% in the cervical tissue. In alternative embodiments, the mechanical stiffness of a cervical tissue can be determined by measuring the surface displacement of the cervix upon application of a specified mechanical stress, e.g., ranging from 0.1 kilopascal to about 500 kilopascals or from about 1 kilopascal to about 100 kilopascals, or from about 5 kilopascals to about 50 kilopascals.

Accordingly, in some embodiments, the method described herein can increase the level of mechanical stress required to produce a certain compressive strain (e.g., about 25%) in a cervical tissue, upon injection and gelation of the silk fibroin-based composition within the cervical tissue, by at least about 30%, at least about 40%, at least about 50%, at least about 60%, at least about 70%, at least about 80%, at least about 90%, at least about 100%, at least about 200%, at least about 300%, at least about 400%, at least about 500% or more, as compared to that of a cervical tissue in the absence of the silk fibroin-based composition. In alternative embodiments, upon injection and gelation of the silk fibroin-based composition within a cervical tissue, the method described herein can decrease the surface displacement of the injected cervical tissue under a specified mechanical stress, by at least about 30%, at least about 40%, at least about 50%, at least about 60%, at least about 70%, at least about 80%, at least about 90%, at least about 95%, at least about 97% or more, as compared to that of a cervical tissue in the absence of the silk fibroin-based composition. In one embodiment, the method described herein can completely inhibit the surface displacement of an injected cervical tissue under a constant stress, as compared to that of a cervical tissue in the absence of the silk fibroin-based composition.

The relationship of stress and strain can be characterized by a Young's modulus or an elastic modulus, which can be defined by the slope of the linear portion of a stress-strain curve that results from conventional mechanical testing protocols, e.g., the mechanical set-up described in FIG. 5A.

Hence, in some embodiments, the method described herein can increase the Young's modulus or elastic modulus of an injected cervix tissue by at least about 30%, at least about 40%, at least about 50%, at least about 60%, at least about 70%, at least about 80%, at least about 90%, at least about 100%, at least about 200%, at least about 300%, at least about 400%, at least about 500% or more, as compared to that of a cervical tissue in the absence of the silk fibroin-based composition.

In still other embodiments, the mechanical stiffness of a cervical tissue can refer to mechanical strength of a cervical tissue to maintain a closed cervix for a period of time (e.g., during pregnancy) while holding a fetus inside a uterus. The phrase “closed cervix” as used herein refers to a cervical opening of less than 10 mm, less than 9 mm, less than 8 mm, less than 7 mm, less than 6 mm, less than 5 mm, less than 4 mm, less than 3 mm, less than 2 mm, less than 1 mm or smaller. In some embodiments, a closed cervix can refer to a cervix with a cervical opening undetectable by bare eyes and/or instruments. Accordingly, in such embodiments, upon injection and gelation of the silk fibroin-based composition within a cervical tissue, the methods described herein can decrease the size of a cervical opening by at least about 30%, at least about 40%, at least about 50%, at least about 60%, at least about 70%, at least about 80%, at least about 90%, at least about 95%, at least about 97% or more, as compared to that of a cervical tissue in the absence of the silk fibroin-based composition. In some embodiments, the method described herein can decrease the size of the cervical opening to less than 10 mm, less than 9 mm, less than 8 mm, less than 7 mm, less than 6 mm, less than 5 mm, less than 4 mm, less than 3 mm, less than 2 mm, less than 1 mm or smaller. In some embodiments, the methods described herein can maintain a cervical opening of no greater than 10 mm (e.g., no greater than 9 mm, no greater than 8 mm, no greater than 7 mm, no greater than 6 mm, no greater than 5 mm or less) for a period of time (e.g., during pregnancy, or until a gestational age reaches at least about 23 weeks, at least about 24 weeks, at least about 25 weeks, at least about 26 weeks, at least about 27 weeks, at least about 28 weeks, at least about 29 weeks, at least about 30 weeks, at least about 31 weeks, at least about 32 weeks, at least about 33 weeks, at least about 34 weeks, at least about 35 weeks, at least about 36 weeks, at least about 37 weeks, at least about 38 weeks, at least about 39 weeks, at least about 40 weeks, at least about 41 weeks, at least about 42 weeks, or longer.)

In some embodiments, upon injection and gelation of the silk fibroin-based composition within a cervical tissue, the method described herein can decrease rate of undesirable cervical opening (e.g., due to a weakened cervix during pregnancy) by at least about 30%, at least about 40%, at least about 50%, at least about 60%, at least about 70%, at least about 80%, at least about 90%, at least about 95%, at least about 97% or more, as compared to that of a cervical tissue in the absence of the silk fibroin-based composition. Stated another way, upon injection and gelation of the silk fibroin-based composition within a cervical tissue, the method described herein can extend the length of a gestational age by at least about 1 month, at least about 2 months, at least about 3 months, at least about 4 months, at least about 5 months, at least about 6 months, least about 7 months, at least about 8 months, at least about 9 months or longer, as compared to that of a cervical tissue in the absence of the silk fibroin-based composition. Accordingly, some embodiments of the methods described herein can be used to reduce likelihood of a preterm birth or a miscarriage, e.g., by at least about 10%, at least about 20%, at least about 30%, at least about 40%, at least about 50%, at least about 60%, at least about 70%, at least about 80%, at least about 90% or more, as compared to the likelihood of a preterm birth or a miscarriage in the absence of the silk fibroin-based composition.

Methods for measuring or determining mechanical stiffness of a cervical tissue or determining the size of a cervical opening in vivo are well known in the art, for example, including, but not limited to, elastography, ultrasound imaging, magnetic resonance elastography, computed tomography, colposcopy and any combinations thereof. Additional methods and/or devices that can be used for measuring or determining the mechanical stiffness of a cervical tissue and/or determining the size of a cervical opening in vivo are disclosed in the U.S. Pat. App. Nos. US 2011/0190579; US 2010/0222679; and US 2007/0167819; US 2004/0210136; as well as U.S. Pat. Nos.: 6,101,408; 5,791,346; 7,947,001; and 5,842,995, the contents of which are incorporated herein by reference in their entireties. In vivo devices for measuring cervical distensibility and mechanical aspirators to assess uterine cervix in vivo can also be used for mechanical property assessment of a cervical tissue. See, e.g., Cabrol D. et al., 1990 Gynecol. Obstet. Invest. 29: 32; and Mazza E. et al. et al., 2006 Med. Image Anal., 10: 125, respectively. In some embodiments, the mechanical stiffness of a cervical tissue can be measured ex vivo or in vitro, e.g., using a uniaxial compression test described in Example 1 and FIGS. 5A-5C.

Mechanical properties of a silk fibroin-based gel formed in a cervix can be modulated by altering the amount of beta-sheets formed in the silk fibroin. Accordingly, in some embodiments, the method described herein can further comprise increasing amount of beta sheet in the silk fibroin-based composition, upon injection into a cervical tissue. In some embodiments, the method described herein can further comprise inducing beta-sheet formation in the silk fibroin-based composition, upon injection into a cervical tissue. Methods for modulating (e.g., increasing) the amount of beta-sheets in the silk fibroin-based gel are well known in the art, including, but not limited to, controlled slow drying (see, e.g., Lu et al., 10 Biomacromolecules 1032 (2009)), water annealing (see, e.g., Jin et al., Water-Stable Silk Films with Reduced β-Sheet Content, 15 Adv. Funct. Mats. 1241 (2005)), stretching (see, e.g., Demura & Asakura, Immobilization of glucose oxidase with Bombyx mori silk fibroin by only stretching treatment and its application to glucose sensor, 33 Biotech & Bioengin. 598 (1989)), compressing, solvent immersion, including methanol (see, e.g., Hofmann et al., 2006), ethanol (see, e.g., Miyairi et al., 1978), glutaraldehyde (see, e.g., Acharya et al., 2008) and 1-ethyl-3-(3-dimethyl aminopropyl) carbodiimide (EDC) (see, e.g., Bayraktar et al., 2005); pH adjustment (see, e.g., U.S. Patent App. No. US2011/0171239, the content of which is incorporated herein by reference), heat treatment, shear stress (see, e.g., International App. No.: WO 2011/005381, the content of which is incorporated herein by reference), sonication (see, e.g., U.S. Pat. App. No. U.S. 2010/0178304 and International App. No. WO 2008/150861, the contents of which are incorporated herein by reference), and any combinations thereof.

In some embodiments, the silk fibroin-based composition can be exposed, upon injection into a cervix, to an alcohol treatment (e.g., methanol or ethanol) or a water-annealing treatment. In some embodiments, the silk fibroin-based composition can be exposed to an alcohol treatment (e.g., methanol or ethanol) or a water-annealing treatment after initial gelation of the silk fibroin-based composition.

In some embodiments, post-treatment of the silk fibroin-based gel, e.g., water-annealing or solvent immersion (e.g., alcohol treatment), can be used to control the release of an active agent from the silk fibroin-based gel injected into the cervix. In some embodiments, post-treatment of the silk fibroin-based gel, e.g., water-annealing or solvent immersion (e.g., alcohol treatment), can be used to modulate the degradation or solubility properties of the silk fibroin-based gel.

In some embodiments, the methods can further comprising administering an active agent described herein to a subject. For example, in one embodiment, the methods can further comprising injecting at least one active agent described herein into at least a portion of a cervix as described herein. The active agents can be injected prior to, concurrently with, or after administration of the silk fibroin based composition into the cervix. In some embodiments where the active agents are injected concurrently with the silk fibroin-based composition, the active agents can be blended with the silk fibroin-based composition.

The silk fibroin-based composition can be injected into the cervix at any time during pregnancy. In some embodiments, the silk fibroin-based composition can be injected into a cervix between about 1 week and about 32 weeks of gestational age, between about 4 weeks and about 30 weeks of gestational age, or between about 5 weeks and about 25 weeks of gestational age. In some embodiments, the silk fibroin-based composition can be injected into the cervix before 1 week of gestational age. In other embodiments, the silk fibroin-based composition can be injected into the cervix after 32 weeks of gestational age. In some embodiments, the silk fibroin-based composition can be injected into a cervix before any suspected or a potential miscarriage or pregnancy loss. The term “gestational age” as used herein refers to the age of an embryo or fetus (or newborn infant). Some common methods of calculating gestational age include counting either from the first day of the woman's last menstrual period (LMP), or from 14 days before conception (fertilization). Counting from the first day of the LMP involves the assumption that conception occurred 14 days later. If the day of conception is known, the 14th day before conception can be used. In addition to the aforementioned methods, any other art-recognized methods known to a skilled practitioner can be used to calculate the gestational age.

In some embodiments, the methods described herein can be performed in a subject any time before fetal viability, e.g., the ability of a fetus to survive outside the uterus, or before the third trimester.

In other embodiments, the silk fibroin-based composition can be injected into a cervix prior to pregnancy.

In some embodiments, the methods described herein can be used as an adjunct therapy to a cerclage procedure and/or progesterone therapy. The methods described herein can be used prior to, concurrently with, or after a cerclage procedure and/or progesterone therapy.

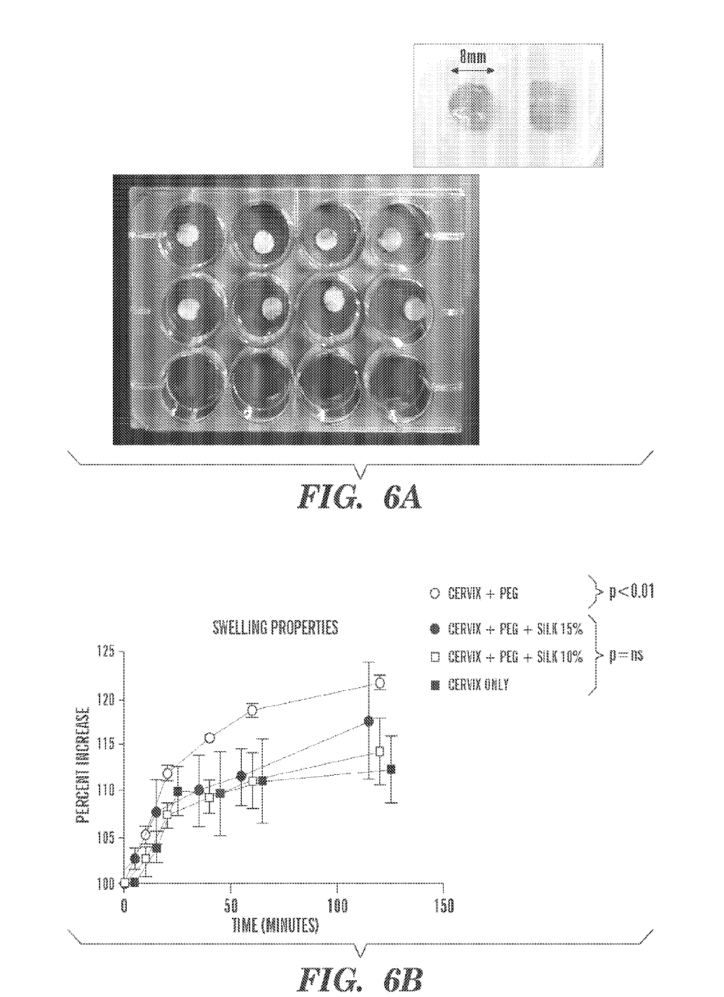

In some embodiments, the method described herein can provide an injected cervical tissue with comparable swelling behavior in a specified condition (e.g., in a buffered solution such as PBS) as a native cervical tissue or an untreated cervical tissue. The term “comparable” as used herein refers to a difference in swelling behavior between an injected and an untreated cervical tissue of less than 20%, less than 10%, less than 9%, less than 8%, less than 7%, less than 6%, less than 5%, less than 4%, less than 3%, less than 2%, less than 1% or smaller. The swelling behavior can be determined by any methods known in the art, e.g., by measuring a change in size of a cervical tissue over a period of time in a specified condition (e.g., immersion in a buffered solution) as shown in FIGS. 6A-6B. The methods and/or devices for determining or monitoring the cervical opening size as described earlier can also be adapted for use in determining or monitoring the swelling behavior of a cervical tissue in vivo.

Without wishing to be limited, the silk fibroin-based composition can be injected into any other tissues in need of increased mechanical stiffness, e.g., increased loading-bearing support. In some embodiments, other tissues in need of increased mechanical stiffness includes, but not limited to, incompetent tissues (e.g., “weakened” tissue or “softened” tissue) or dilated tissue structures such as valve repair, gastro-esophageal sphincter, bladder sphincter, bladder neck or an anal sphincter.

### Exemplary Methods to Initiate Gelation of a Silk Fibroin-Based Composition

In accordance with various embodiments of the methods described herein, a silk fibroin-based composition can form a gel within a period of time upon injection into a cervix of a subject. In some embodiments, the silk fibroin-based composition can form a gel within 6 hours, 5 hours, 4 hours, 3 hours, 2 hours, 1 hour, 50 seconds, 40 seconds, 30 seconds, 20 seconds, 10 seconds, 5 seconds, 1 second or less, upon injection into a cervix of a subject. In such embodiments, the silk fibroin-based composition that has formed a gel can still undergo further molecular changes, e.g., formation of beta-sheets in silk fibroin proteins to provide further stiffness to the cervical tissue. Such molecular changes can be on a different time scale from that of the initial gel formation. The term “gel” or “gelation” as used herein refers to a network of molecules formed through linkages, e.g., crosslinking or ionic interactions, which transform a liquid (including any fluid compositions that is viscous but injectable) into a semi-solid or solid. The term “semi-solid” as used herein refers to structures that in the absence of a confined space (e.g., a rigid space or a rigid container) can keep the shape in which they have been molded or formed for a long period of time, e.g., from hours to days to weeks to months. However, in contrast to solids, semi-solids can be easily deformed (high compliance) and/or often exhibit viscoelastic behavior in shear deformation.

Various approaches can be utilized to cause or facilitate a silk fibroin-based composition to form a gel upon injection, e.g., including, without limitation, addition of multi-component gelation system in the silk fibroin-based composition, or using any art-recognized gelation-inducing agent to initiate gelation of silk fibroin.

In some embodiments, a silk fibroin-based composition can include additional components that will facilitate or accelerate gel formation upon injection of the silk fibroin-based composition into a cervix. In such embodiments, the silk fibroin-based composition can comprise at least two functionally-activated PEG components capable of reacting with one another to form a crosslinked gel, and silk fibroin capable of forming beta-sheets to further stabilize the crosslinked gel. Typically, two or more functionally-activated PEG components in a silk fibroin-based composition are not pre-mixed together during storage or prior to use. In some embodiments, each of the functionally-activated PEG components can be blended with silk fibroin and separated for storage or prior to use. At the time of injection (including immediately before, during, or after the injection), two or more functionally-activated PEG components in the silk fibroin-based composition can then be mixed together such that the functionally-activated PEG components can react with one another to form a crosslinked gel upon injection into the cervix, and silk fibroin can further form beta-sheets to further stabilize the crosslinked gel.

In alternative embodiments, two or more functionally-activated PEG components and the silk fibroin can be injected independently and separately into a cervix.

Functionally-activated PEG components to be injected into a cervix can each be independently present at any concentrations or amounts. In some embodiments, functionally-activated PEG components to be injected into a cervix can each be independently blended into a silk fibroin solution at any concentrations or amounts. For example, functionally-activated PEG components to be injected into a cervix can each be independently present in a concentration of about 1% (w/v) to about 30% (w/v), or about 3% (w/v) to about 25% (w/v), or about 5% (w/v) to about 20% (w/v), or about 7.5% (w/v) to about 15% (w/v). In one embodiment, functionally-activated PEG components to be injected into a cervix can each be independently present in a concentration of about 10% (w/v).

In some embodiments, the weight ratio of silk fibroin to a functionally-activated PEG component in a silk fibroin-based composition can range from about 1:100 to about 100:1, or from about 1:10 to about 10:1, or from about 1:3 to about 5:1, or from about 1:2 to about 2:1. In some embodiments, silk fibroin and a functionally-activated PEG component can be present in a silk fibroin-based composition at a ratio of about 1:1. In some embodiments, silk fibroin and a functionally-activated PEG component can be present in a silk fibroin-based composition at a ratio of about 7:10.

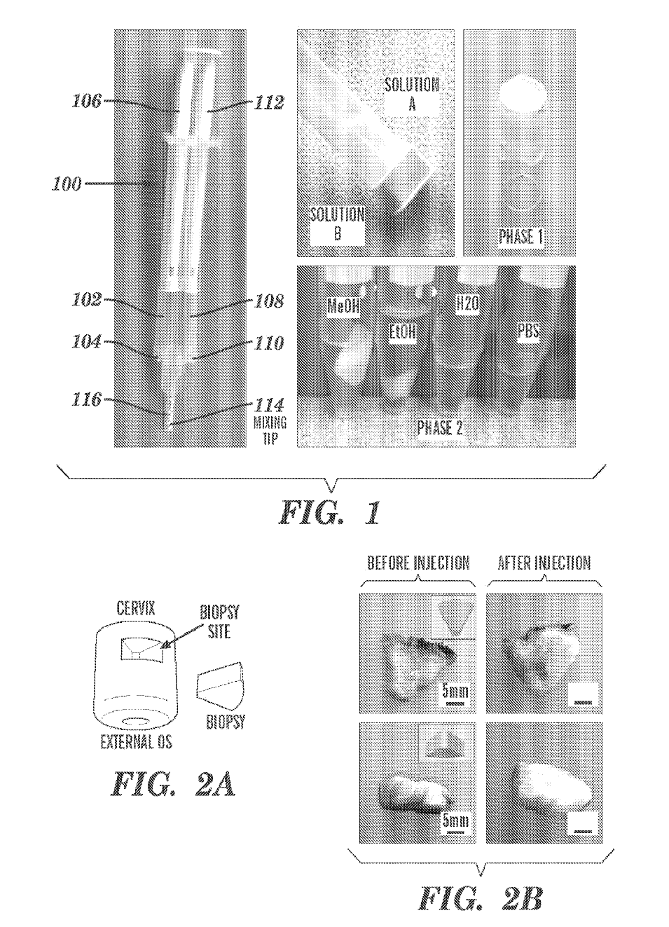

Without wishing to be bound by theory, the crosslinking reactions between two or more functionally-activated PEG components generally contribute to an initial gelation of the silk fibroin-based composition (FIG. 1: Phase 1). Such initial gelation can occur within seconds, e.g., less than 60 seconds, less than 50 seconds, less than 40 seconds, less than 30 seconds, less than 20 seconds, less than 10 seconds, less than 5 seconds, less than 1 second or shorter. Further stiffening of the silk fibroin-based gel can occur through beta-sheet formation in silk fibroin, e.g., by further exposing the silk fibroin-based gel to a post-treatment, e.g., comprising alcohol immersion as discussed earlier. The additional stiffening process (e.g., silk beta-sheet formation) can occur over a period of time longer than an initial gelation due to PEG cross-linkages.

Functionally activated PEG components: Each of the PEG components is activated with one or more functional groups. The term “activated PEG components” refers to PEG components which have been chemically modified to have two or more functional groups that are capable of chemically reacting with the other functional groups of the same or different PEG component to form covalent bonds, thereby forming a crosslinked matrix. PEGs components herein are typically multifunctionally activated, i.e., containing two or more functional groups (e.g., difunctionally activated, tetrafunctionally activated, or star-branched).

At least one of the PEG components can be a multi-arm PEG derivative (e.g., 2-arm, 4-arm, 8-arm, and 12-arm, etc.). In some embodiments, each of the PEG components can be a multi-arm PEG derivative (e.g., 2-arm, 4-arm, 8-arm, and 12-arm, etc.). The term “multi-arm PEG derivatives” described herein refers to a branched poly(ethylene glycol) with at least about 2, at least about 4, at least about 6, at least about 8, at least about 12 PEG polymer chains or derivatives thereof (“arms”) or more. Multi-arm or branched PEG derivatives include, but are not limited to, forked PEG and pendant PEG. An example of a forked PEG can be represented by PEG-YCHZ₂, where Y is a linking group and Z is an activated terminal group linked to CH by a chain of atoms of defined length. The International Application No. WO 99/45964, the content of which is incorporated herein by reference, discloses various forked PEG structures that can be used for some embodiments described herein. The chain of atoms linking the Z functional groups to the branching carbon atom can serve as a tethering group and can comprise, for example, alkyl chains, ether chains, ester chains, amide chains and combinations thereof. A pendant PEG can have functional groups, such as carboxyl, covalently attached along the length of the PEG segment rather than at the end of the PEG chain. The pendant reactive groups can be attached to the PEG segment directly or through a linking moiety, such as alkylene. Additional multi-arm or branched PEG derivatives such as the ones disclosed in the U.S. Pat. No. 5,932,462, the content of which is incorporated herein by reference, can be also used for the purpose of different embodiments and/or aspects described herein. In some embodiments, the multi-arm PEG derivatives can encompass multi-arm PEG block copolymer, e.g., but not limited to, 8-arm (PPO-PEG) block copolymer and 8-arm (PLA-PEG) block copolymer. Methods for producing such multi-arm PEG block copolymer are well known in the art. See, for example, the U.S. Patent Application No. US 2005/0147681, for exemplary multi-arm PEG block copolymer and methods of making the same.

In some embodiments of a silk fibroin-based composition described herein, each of the PEG components can have the same or different number of arms. Multi-arms of PEG derivatives, for example, PEG derivatives with at least 4 arms, are typically more efficient for crosslinking reaction. The number of crosslinks or mechanical properties of the crosslinked polymer matrix described herein can be modulated by the number of PEG arms and/or functional groups. In one embodiment, 4-arm PEG derivative is used to form silk-PEG crosslinked matrix. In another embodiment, 8-arm PEG derivative is used to form silk-PEG crosslinked matrix. In some embodiments, The PEG component can also be a combination of PEG derivatives with different arm numbers. Different arms of the PEG component can carry the same or different numbers or types of functional groups.

Various functional groups can be used to activate the PEG component for crosslinking reaction. As described herein, “functional group A” and “functional group B” are generally used to refer to a pair of functional groups capable of chemically reacting with one another and hence are used for activating PEG components for crosslinking reaction. For example, functional group A can be —NH₂, thiol (—SH), —S⁻, —OH, —PH₂, —CO—NH—NH₂, and any combinations thereof; and functional group B can be —NHS, acrylate, vinyl sulfone, maleimide, —CO₂N(COCH₂)₂, —CO₂H, —CHO, —CHOCH₂, N═C═O, —SO₂CH═CH₂, —N(COCH)₂, —S—S—(C₅H₄N), and any combinations thereof. Other functional groups known to the skilled in the art can also be used. In one embodiment, the pair of functional groups in the PEG components includes thiol/maleimide. In one embodiment, the pair of functional groups in the PEG components includes thiol/acrylate. In another embodiment, the pair of functional groups in the PEG components includes amine/N-hydroxysuccinimide. In some embodiments, the pair of PEG components used herein includes multi-arm PEG-thiol and multi-arm PEG-maleimide. In one embodiment, the pair of PEG components used herein includes 4-arm PEG-thiol and 4-arm PEG-maleimide.

The ratio of different functionally activated PEG components present in a silk fibroin-based composition can depend on the number of functional groups in each PEG component. By way of example only, two functionally activated PEG components can be combined in a ratio ranging from about 10:1 to about 1:10, inclusive, or from about 5:1 to about 1:5, inclusive. In some embodiments, one PEG component can be present in excess after crosslinking reaction. In one embodiment, the two functionally activated PEG components can be combined in a ratio of about 1:1. One of skill in the art can determine the ratio of different functionally activated PEG components based on reaction stoichiometry and types of chemical reactions.

The reaction of the functionally activated PEGs in forming a crosslinked network can occur by a number of different chemical reactions depending on the functionality of the groups attached to the PEGs. For example, the gel can be formed by a Michael-type addition reaction or a condensation reaction. In general, a Michael-type addition reaction involves the reaction of an α,β-unsaturated carbonyl with a nucleophile. A Michael-type addition reaction can occur at a pH 6 or greater, e.g., pH 6, pH 7, pH 8, pH 9 or higher. Michael addition reactions are well known by those skilled in the art. Examples of moieties on functionalized PEGs which can undergo a Michael's addition reaction include, but are not limited to: PEG-SH combined with PEG-maleimide; and PEG-SH combined with PEG-acrylate. In some embodiments, the reaction could be activated with a buffer with a pH greater than about 4, by a catalytic amount of various amines or a combination thereof. A condensation reaction is a chemical reaction in which two molecules or moieties react and become covalently bonded to one another by the concurrent loss of a small molecule, often water, methanol, or a type of hydrogen halide such as hydrogen chloride. In polymer chemistry, a series of condensation reactions can take place whereby monomers or monomer chains add to each other to form longer chains. Examples of functional groups on activated PEGs which can undergo a condensation reaction include, but are not limited to, PEG-NHS ester and PEG-NH₂. Without wishing to be bound by theory, a Michael addition reaction can contribute to a longer stability of the resulting crosslinked network since thioether bonds are formed as compared to the more hydrolytically labile thioester bonds formed from the reaction of thiols with activated esters.

Each of the PEG components in a silk fibroin-based composition can be independently provided as a powder, a suspension or a solution, or one component is provided as a powder and another component is provided as a suspension or a solution. Similarly, silk fibroin in a silk fibroin-based composition can also be provided as a powder, a suspension or a solution. In one embodiment, all the components in a silk fibroin-based composition are powders. In one embodiment, at least one component is suspended or dissolved in an aqueous solution. In one embodiment, at least the silk fibroin is provided in an aqueous solution. In one embodiment, the silk fibroin is dissolved or suspended in water to prepare a silk fibroin solution. In another embodiment, the PEG component can be suspended or dissolved in a silk fibroin solution.

The components of a silk fibroin-based composition (e.g., PEG components and/or silk fibroin) can be individually prepared and stored in an acidic, neutral or basic solution (i.e., at any pHs). Prior to combining the components into one composition to form a crosslinked polymer matrix, the pH of the components can be each adjusted to a desired pH for crosslinking reaction, e.g., at pH 6 or greater, including pH 7, pH 8, pH 9 or greater Alternatively, the final pH of a silk fibroin-based composition can reach pH 6 or higher, including pH 7, pH 8, pH 9 or greater after all the components are combined together. Therefore, at least one component can be prepared in an acidic solution, while the other can be prepared in a basic or neutral solution such that the combination results in a desirable pH, e.g., pH 6, pH 7, pH 8, pH9 or higher.

In various embodiments, the molecular weight of each of the PEG components or other synthetic polymers can independently vary depending on the desired application. In some embodiments, the molecular weight (MW) is about 100 Da to about 100000 Da, about 1000 Da to about 20000 Da, or about 5000 Da to about 15000 Da. In some embodiments, the molecular weight of the PEG components is about 10,000 Da. Further details about gel formation of a silk fibroin-based composition comprising PEG components can be found in the International Patent Application No. PCT/US11/50238 (WO 2012/031144), the contents of which are incorporated herein by reference.

To facilitate injection of a silk fibroin-based composition comprising at least at least two functionally-activated PEG components as described herein into a cervical tissue of a subject, in some embodiments, the delivery device described in International App. No. WO 2012/031144, the content of which is incorporated herein by reference, can be used to perform one or more embodiments of the methods described herein.

Without wishing to be bound, it is contemplated that other multi-component gelation systems (e.g., two-component gelation system) can be used to replace PEG gelation system or be included in a silk fibroin-based composition described herein, provided that silk fibroin can form beta sheet and stabilize the crosslinked network. Examples of two-component gelation systems include, but are not limited to, alginate construct systems, fibrin glues (e.g., fibrinogen and thrombin) and fibrin glue-like systems, self-assembled peptides, synthetic polymer systems (e.g., PEG system) and any combinations thereof.

In some embodiments, the two-component gelation system can include fibrin glue. Fibrin glue consists of two main components, fibrinogen and thrombin. When combined in equal volumes, thrombin converts the fibrinogen to fibrin by enzymatic action at a rate determined by the concentration of thrombin. The result is a biocompatible gel which gelates between about 5 to about 60 seconds. In such embodiments, the silk fibroin can further stabilize the fibrin gel through silk beta-sheet formation. Examples of fibrin glue-like systems include, but are not limited to, Tisseel™ (Baxter), Beriplast P™ (Aventis Behring), Biocol® (LFB, France), Crosseal™ (Omrix Biopharmaceuticals, Ltd.), Hemaseel HMN® (Haemacure Corp.), Bolheal (Kaketsuken Pharma, Japan) and CoStasis® (Angiotech Pharmaceuticals).

In some embodiments, a two-component gelation system can include a synthetic polymer system. In addition to PEG as described herein, other examples of synthetic polymers can include, but are not limited to, polyamino acids, polysaccharides, polyalkylene oxide and any combinations thereof. In these synthetic polymer systems, at least two components of the synthetic polymer system can be functionally activated using reaction chemistry known in the art such that these two components of the synthetic polymer system can react with each other to form a crosslinked network.

Another approach to form a silk fibroin-based gel upon injecting a silk fibroin-based composition into a cervix can involve exposure of a silk fibroin-based composition to a gelation-inducing agent for a sufficient period of time. For example, in some embodiments, a gelation-inducing agent can be administered concurrently with injection of a silk fibroin-based composition into a cervical tissue. In other embodiments, a gelation-inducing agent can be administered upon injection of a silk fibroin-based composition into a cervical tissue. In such embodiments where a gelation-inducing agent is added, functionally activated PEG components described herein need not be included in a silk fibroin-based composition (e.g., in one embodiment, functionally activated PEG components are excluded from a silk fibroin-based composition that is subjected to exposure of a gelation-inducing agent, while in another embodiment, a silk fibroin-based composition comprises at least two functionally activated PEG components, whether the silk fibroin-based composition comprises a gelation-inducing agent or not). The term “gelation-inducing agent” as used herein refers to any agent (e.g., a molecule, a compound, or a stimulus) that can induce or initiate gelation of a silk fibroin-based composition, e.g., without limitations, heat, light, electron beams, redox reagents, ultrasound, electric field (or voltage), acidic solutions, other initiators, and any combinations thereof. In some embodiments, a gelation-inducing agent can induce or initiate gelation of silk fibroin in a silk fibroin-based composition. Gelation-inducing agents that can induce or initiate silk fibroin gelation include, but are not limited to, ultrasound, electric field (or voltage), acidic solutions, and any combinations thereof.

In some embodiments, a gelation-inducing agent includes ultrasound. The use of ultrasound, sonication or ultrasonication (an act of applying ultrasound energy to a material) to initiate silk fibroin gelation has been described in U.S. patent application Ser. No. 12/601,845 (which is a 35 U.S.C. § 371 National Stage of International Application No. PCT/US2008/065076 filed on May 29, 2008, which claims the benefit of priority under 35 U.S.C. § 119(e) of U.S. Provisional Application No. 60/940,554, filed May 29, 2007), the contents of which are herein incorporated by reference, and any embodiments described in those disclosures can be adopted for use in the methods described herein.

In some embodiments, a silk fibroin-based composition can be subjected to a treatment comprising ultrasound for a sufficient period of time to initiate gelation prior to, during, or after injection of the silk fibroin-based composition into a cervix. In some embodiments, ultrasound can be administered concurrently with injection of a silk fibroin-based composition into the cervix of a subject. In some embodiments, ultrasound can be administered upon injection of a silk fibroin-based composition into the cervix of a subject. Gelation typically begins when a silk fibroin-based composition is exposed to ultrasound and continues for a period of time even after the ultrasound treatment ends. Thus, in some embodiments, a silk fibroin-based composition can be subjected to a treatment comprising ultrasound or sonication, prior to injection of the silk fibroin-based composition into a cervix, where the duration and/or amplitude of ultrasound applied to the silk fibroin-based composition can be adjusted to enable the silk fibroin-based composition to initiate gelation but to remain in an injectable state until injection of the silk fibroin-based composition into a cervix is completed.

Typically, ultrasonication or ultrasound treatment can last from about 5 seconds to about 60 seconds or more, depending on the amount of silk fibroin used, the concentration of the silk fibroin solution, and other factors appreciated by those of ordinary skill in the art. For example, the ultrasonication or ultrasound treatment can last from about 15 seconds to about 45 seconds.

The amplitude of the ultrasound applied to a silk fibroin-based composition and/or the concentration of the silk fibroin can be adjusted for desirable gelation time. For example, the ultrasound amplitude can range from about 25% to about 35% power output (typically, about 7 watts to about 10 watts) when the concentration of the silk fibroin ranges from about 10 wt % to about 15 wt %. In another embodiment, the amplitude can range from about 25% to about 55% power output (typically, about 7 watts to about 21 watts) when the concentration of the silk fibroin is lower, e.g., ranging from about 5% to about 10% (w/v). Those of ordinary skill in the art will be readily able to alter the amplitude of the ultrasound and/or the concentration of the silk fibroin solution to produce a desired degree of gelation and a desired time frame in which gelation occurs.

Cervical ultrasound has been commonly performed during pregnancy. Such art-recognized methods and/or associated transducers can be readily adapted for use in delivering ultrasound energy to a silk fibroin-based composition injected into a cervix as described herein. Without wishing to be bound, methods for monitoring and/or measuring cervix with ultrasound, e.g., described herein U.S. Pat. App. No. US 2006/0089570 and U.S. Pat. No. 5,713,237 1 can also be adapted for use to apply ultrasound to a silk fibroin-based composition as described in some embodiments of the methods provided herein. Any art-recognized cervical or tranvaginal or endovaginal ultrasound transducers or probes, including the ones described in, e.g., but not limited to, U.S. Pat. Nos. 5,222,485, 5,351,692, 4,535,759, and U.S. Pat. App. No. US 2011/0082375 can also be adapted for use in applying ultrasound to a silk fibroin-based composition as described in some embodiments of the methods provided herein.

In alternative embodiments, a gelation-inducing agent includes an electric field (or voltage). The use of an electric field to initiate silk fibroin gelation has been discussed in U.S. patent application Ser. No. 12/974,796 (which is a Continuation-In-Part of PCT application Ser. No. PCT/US2009/058534, filed Sep. 28, 2009, which claims the benefit of priority under 35 U.S.C. § 119(e) of U.S. Provisional Patent Application No. 61/100,352 filed Sep. 26, 2008, and U.S. Provisional Patent Application No. 61/156,976 filed Mar. 3, 2009), the contents of which are incorporated herein by reference, and any embodiments described therein can be used for the purpose of various aspects and embodiments described herein.

Thus, in some embodiments, upon injection into a cervix, the silk fibroin-based composition can be exposed to a treatment comprising an electric field for a sufficient period of time to initiate gelation. In some embodiments, a treatment comprising an electric field can be administered concurrently with injection of a silk fibroin-based composition into the cervix of a subject. Depending on voltages applied, a silk fibroin-based composition can be exposed to a treatment comprising an electric field for seconds, minutes, or hours. Generally, a silk fibroin-based composition can be exposed to an electric field for a shorter duration when a higher voltage is applied, and for a longer duration when a lower voltage is applied. Thus, in some embodiments, a silk fibroin-based composition can be exposed to a treatment comprising an electric field for about 15 seconds to about 1 hour, about 30 seconds to about 30 minutes, or about 1 minute to about 15 minutes. In other embodiments, a silk fibroin-based composition can be exposed to a treatment comprising an electric field for about 30 minutes to about 6 hours, about 1 hour to about 5 hours, or about 1.5 hour to about 4 hours.

In some embodiments, an electric field applied to a silk fibroin-based composition can generate a voltage of about 5V to about 100V, about 10 V to about 100 V, or about 20 V to about 80V. Without wishing to be bound, a voltage below 1V can also be used in the method described herein. In some embodiments, the voltage is a DC voltage. Those of ordinary skill in the art can readily alter, without undue experimentation, duration and/or voltage of an electric field, and/or the concentration of a silk fibroin solution, to produce a desired degree of gelation and/or a desired time frame in which gelation occurs.

It can be desirable to convert such electrogelated silk fibroin-based gel (e.g., formation of a silk fibroin-based gel by applying an electric field to a silk-fibroin based composition) to a silk fibroin-based solution at a later time point, e.g., when the pregnancy has gone to term (e.g., it is about time to deliver a baby or during labor), pre-term labor begins, or pre-term or premature rupture of amniotic membrane occurs, or any condition where reducing mechanical stiffness of a cervical tissue previously treated with an electrogelated silk fibroin-based gel is desirable. In such embodiments, the electrogelated silk fibroin gel previously formed in the cervix can be solubilized by exposing it to an electric field with a reversed polarity (e.g., reversal of a polarity that was previously applied to form a silk fibroin-based gel).

Cervical electrodes have been commonly used to excise a tissue from a cervix or for cervix scanning, and thus such art-recognized cervical electrodes can be readily adapted for use in delivering a voltage or an electric field to a cervix. Examples of cervical electrodes and methods of delivering a voltage to a cervix that can be used for the methods described herein include, but are not limited to, U.S. Pat. Nos. 6,641,581; 6,123,701; 7,033,355; and U.S. Pat. App. Nos. US 2009/0318914 and US 2009/0137925, the contents of which are incorporated herein by reference.

Other gelation-inducing agents can include a salt solution and/or an acid solution. Salt solutions are known in the art to assist in inducing gelation of silk fibroin. Typical salt solutions containing ions of potassium, calcium, sodium, magnesium, copper, and/or zinc can be used. Thus, a silk fibroin-based composition can further comprise a salt solution (e.g., sodium salts) or be exposed to a salt solution (e.g., sodium salts) when the silk fibroin-based composition is injected into the cervix. Alternatively, gelation of silk fibroin can be induced by adjusting the pH of a silk fibroin-based composition. pH-induced silk fibroin-based gels and formation thereof are described in U.S. patent application Ser. No. 12/974,796 filed Dec. 21, 2010, the content of which is incorporated herein by reference. In particular, reducing the pH of a silk fibroin-based composition (e.g., ˜pH 4 or lower) upon injection can initiate gelation of silk fibroin. In such embodiments, a silk fibroin-based composition can further comprise an acidic solution or be exposed to an acidic solution when the silk fibroin-based composition is injected into the cervix.

### Silk Fibroin-Based Compositions

The term “silk fibroin-based composition” refers to a composition comprising silk fibroin in any concentrations or amounts. Silk fibroin is generally known as a naturally derived, fibrous protein that displays remarkable mechanical properties, chemical versatility and biocompatibility (13). The mechanical properties of silk fibers arise from the self-assembly of many small crystalline β-sheet structures via intramolecular and intermolecular hydrogen bonding in combination with less organized but hydrogen-bonded domains (14, 15). Gelation of silk fibroin solution can be induced, e.g., by sonication, physical agitation, alcohol dehydration and electrical current (16-18). In addition, the physical properties of silk fibroin-based materials can be tuned to meet functional demands by blending with other macromolecules such as tropoelastin (increased elasticity) (19) or hyaluronan (biological signaling) (20).

As used herein, the term “silk fibroin” includes silkworm fibroin and insect or spider silk protein. See e.g., Lucas et al., 13 Adv. Protein Chem. 107 (1958). Any type of silk fibroin including naturally-derived synthetic silk fibroin may be used according to various aspects described herein. Silk fibroin produced by silkworms, such as Bombyx mori, is the most common and represents an earth-friendly, renewable resource. For instance, silk fibroin may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants (see, e.g., WO 97/08315; U.S. Pat. No. 5,245,012), and variants thereof, that can be used. In some embodiments, silk fibroin for use in the methods described herein can be modified to comprise an active agent or be conjugated to an active agent. Silk sutures have already been used by clinicians in a cerclage therapy, thus silk fibroin is typically biocompatible with cervical tissues.

In some embodiments, silk fibroin can exclude an amphiphilic peptide. “Amphiphilic peptides” possess both hydrophilic and hydrophobic properties. Amphiphilic molecules can generally interact with biological membranes by insertion of the hydrophobic part into the lipid membrane, while exposing the hydrophilic part to an aqueous environment. In some embodiments, the amphiphilic peptide can comprise a RGD motif. An example of an amphiphilic peptide is a 23 RGD peptide having an amino acid sequence: HOOC-Gly-Arg-Gly-Asp-Ile-Pro-Ala-Ser-Ser-Lys-Gly-Gly-Gly-Gly-Ser-Arg-Leu-Leu-Leu-Leu-Leu-Leu-Arg-NH₂. Other examples of amphiphilic peptides include the ones disclosed in the U.S. Patent App. No. US 2011/0008406. Thus, in some embodiments, silk fibroin can exclude the 23 RGD peptide, and/or the amphiphilic peptides described in the U.S. Patent App. No. US 2011/0008406, the contents of which are incorporated herein by reference.

Silk fibroin-based compositions used in the methods described herein can comprise silk fibroin at any concentrations, depending upon the desirable mechanical stiffness of the resultant cervical tissue upon injection and gelation. Using higher concentrations of silk fibroin generally yields a cervical tissue of higher mechanical stiffness than using lower concentrations of silk fibroin. In some embodiments, a silk fibroin-based composition can comprise silk fibroin at a concentration of about 5 wt % to about 30 wt %. In some embodiments, the silk fibroin can have a concentration of about 5 wt % to about 10 wt %. In one embodiment, the silk fibroin can have a concentration of about 7 wt %.

In some embodiments, a silk fibroin-based composition can be formulated such that a silk fibroin-based gel formed upon injection of the silk fibroin-based composition into a cervix can degrade at an optimum rate slow enough to maintain a desirable range of mechanical stiffness over a pre-determined period of time (e.g., over a period of time until pregnancy approaches term, or over a period of time as recommended by a skilled practitioner based on each individual's pregnancy condition), but can reach a relatively lower mechanical stiffness after the pre-determined period of time is over, e.g., in order to facilitate delivery of a newborn.

In some embodiments, a silk fibroin-based composition for cervical support can be formulated to provide a well-defined degradation profile, e.g., a time-dependent degradation profile to meet the requirements of pregnancy. For example, a silk fibroin-based composition for cervical support can be formulated such that in the midtrimester and early third trimester, the composition, upon injection, can stiffen the cervical tissue to prevent cervical shortening, but when it is close to term, the composition can respond by degradation to allow normal cervical dilation.

Degradation rate of a silk fibroin-based gel formed upon injection of a silk fibroin-based composition can be modulated by a number of factors, for example, but not limited to, controlling beta sheet formation in silk fibroin as described herein, concentrations of silk fibroin, concentrations and/or types of functionally-activated PEG components, and/or types of a gelation-inducing agent applied to a silk fibroin-based composition described herein, silk purification process, silk processing method, and any combinations thereof.

In various embodiments, in addition to provide mechanical strength (e.g., load-bearing support) to a cervix upon injection, silk fibroin can be modified for different applications and/or desired mechanical or chemical properties (e.g., to facilitate formation of a gradient of active agent in a silk fibroin-based gel, or to provide controlled release of an active agent from a silk fibroin-based gel). One of skill in the art can select appropriate methods to modify silk fibroins, e.g., depending on the side groups of the silk fibroins, desired reactivity of the silk fibroin and/or desired charge density on the silk fibroin. In one embodiment, modification of silk fibroin can use the amino acid side chain chemistry, such as chemical modifications through covalent bonding, or modifications through charge-charge interaction. Exemplary chemical modification methods include, but are not limited to, carbodiimide coupling reaction (see, e.g. U.S. Patent Application. No. US 2007/0212730), diazonium coupling reaction (see, e.g., U.S. Patent Application No. US 2009/0232963), avidin-biotin interaction (see, e.g., International Application No. WO 2011/011347) and pegylation with a chemically active or activated derivatives of the PEG polymer (see, e.g., International Application No. WO 2010/057142). Silk fibroin can also be modified through gene modification to alter functionalities of the silk protein (see, e.g., International Application No. WO 2011/006133). For instance, the silk fibroin can be genetically modified, which can provide for further modification of the silk such as the inclusion of a fusion polypeptide comprising a fibrous protein domain and a mineralization domain, which can be used to form an organic-inorganic composite. See WO 2006/076711. Additionally, the silk fibroin matrix can be combined with a chemical, such as glycerol, that, e.g., affects flexibility of the matrix. See, e.g., WO 2010/042798, Modified Silk films Containing Glycerol.

In some embodiments, a silk fibroin-based composition can further comprise at least two functionally-activated PEG components (as described herein) capable of reacting with one another to form a crosslinked matrix, where the silk fibroin is capable of forming beta-sheet structures to further stabilize the crosslinked matrix. In one embodiment, one of the functionally-activated PEG components comprises one or more maleimidyl groups. In one embodiment, one of the functionally-activated PEG components comprises one or more thiol groups. In one embodiment, a silk fibroin-based composition can further comprise a first PEG component functionally activated with at least one maleimidyl group and a second PEG component functionally-activated with at least one thiol group.

In some embodiments, silk fibroin can be also mixed with other biocompatible and/or biodegradable polymers to form a mixed polymer composition comprising silk fibroin. One or more biocompatible and/or biodegradable polymers (e.g., two or more biocompatible polymers) can be added to a silk fibroin solution. The biocompatible polymer that can be used herein include, but are not limited to, polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides, polymer, PLA-PGA, polyanhydride, polyorthoester, polycaprolactone, polyfumarate, collagen, chitosan, alginate, hyaluronic acid and other biocompatible and/or biodegradable polymers. See, e.g., International Application Nos. WO 04/062697; and WO 05/012606, the contents of which are incorporated herein by reference.

In some embodiments, at least one active agent described herein can be added to a silk fibroin-based composition prior to injection into a cervix. In some embodiments, the active agent can be dispersed homogeneously or heterogeneously within a silk fibroin-based composition, dispersed in a gradient, e.g., using the carbodiimide-mediated modification method described in the U.S. Patent Application No. US 2007/0212730, the content of which is incorporated herein by reference.

When introducing an active agent such as a therapeutic agent or biological material into a silk fibroin-based composition, other materials known in the art can also be added with the active agent. For instance, it can be desirable to add materials to promote the growth of the active agent (for biological materials), promote the functionality of the active agent after it is released from the silk fibroin-based composition, or increase the active agent's ability to survive or retain its efficacy during the period it is dispersed in the silk fibroin-based composition. Materials known to promote cell growth include cell growth media, such as Dulbecco's Modified Eagle Medium (DMEM), fetal bovine serum (FBS), non-essential amino acids and antibiotics, and growth and morphogenic factors such as fibroblast growth factor (FGF), transforming growth factors (TGFs), vascular endothelial growth factor (VEGF), epidermal growth factor (EGF), insulin-like growth factor (IGF-I), bone morphogenetic growth factors (BMPs), nerve growth factors, and related proteins can be used.

In some embodiments, a silk fibroin-based composition can further comprise a pharmaceutically acceptable carrier. As used herein, the term “pharmaceutically acceptable carrier” refers to a pharmaceutically-acceptable material, composition or vehicle for administration of the silk fibroin, and optionally functionally-activated PEG components and/or an active agent. Pharmaceutically acceptable carriers include any and all solvents, dispersion media, coatings, antibacterial and antifungal agents, and isotonic and absorption delaying agents, which are compatible with the silk fibroin and the activity of the functionally-activated PEG components and active agent, if any, and are physiologically acceptable to the subject. The pharmaceutical formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), suitable mixtures thereof. In some embodiments, the pharmaceutically acceptable carrier can include an aqueous solution. In some embodiments, the pharmaceutically acceptable carrier can be a buffered solution (e.g. PBS). In some embodiments, the pharmaceutically acceptable carrier can be water.

Additionally, various additives which enhance the stability, sterility, and isotonicity of the injectable compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like. The injectable compositions can also contain auxiliary substances such as wetting or emulsifying agents, pH buffering agents, gelling or viscosity enhancing additives, preservatives, colors, and the like, depending upon the preparation desired. Standard texts, such as “REMINGTON'S PHARMACEUTICAL SCIENCE”, 17th edition, 1985, incorporated herein by reference, may be consulted to prepare suitable preparations, without undue experimentation.

Viscosity of silk fibroin-based compositions described herein can be maintained at a selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected, and the desired viscosity for injection. The important point is to use an amount which will achieve the selected viscosity, e.g., addition of such thickening agents into some embodiments of the silk fibroin-based compositions.

Typically, any additives (in addition to the silk fibroin described herein, and/or functionally-activated PEG components, and/or additional active agents) can be present in an amount of 0.001 to 50 wt % dry weight or in a buffered solution. In some embodiments, the active agent can be present in the order of micrograms to milligrams to grams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any pharmaceutical composition to be administered to a subject in need thereof, it is preferred to determine toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan.

In some embodiments, the silk fibroin-based composition described herein can be sterilized. Sterilization methods for biomedical devices are well known in the art, including, but not limited to, gamma or ultraviolet radiation, autoclaving (e.g., heat/steam); alcohol sterilization (e.g., ethanol and methanol); and gas sterilization (e.g., ethylene oxide sterilization).

Silk fibroin-based compositions described herein can be placed into a cervical tissue of a subject by any methods described herein. In some embodiments, a silk fibroin-based composition described herein is an injectable composition, wherein the silk fibroin-based composition forms a gel upon injection into a cervical tissue of a subject. As used herein, the term “injectable composition” generally refers to a composition that can be delivered or administered into a tissue with a minimally invasive procedure. The term “minimally invasive procedure” generally refers to a procedure that is carried out by entering a subject's body through the skin or through a body cavity or an anatomical opening (e.g., vagina), but with the smallest damage possible (e.g., a small incision, injection). Depending on injection locations of a silk fibroin-based composition into a cervix (e.g., lower part vs. upper part of a cervix), in some embodiments, the injectable composition can be administered or delivered into a cervical tissue by injection through vagina. In other embodiments, the injectable composition can be administered or delivered into a cervical tissue through a small incision on the skin (e.g., abdominal skin) followed by insertion of a needle, a cannula, and/or tubing, e.g., a catheter.

As some embodiments of injectable silk fibroin-based compositions can be used to enhance mechanical stiffness or strength of any “incompetent” or “weakened” tissues other than a cervical tissue (e.g., a heart tissue) in a subject, in some embodiments, the injectable compositions can be delivered into a tissue (e.g., a heart tissue) with any art-recognized minimally invasive procedure, e.g., through a small incision on the skin or through a body cavity or an anatomical opening, followed by insertion of a needle, a cannula, and/or tubing, e.g., a catheter. Without wishing to be limited, the injectable composition can be administered or placed into a tissue by surgery, e.g., implantation.

In some embodiments, an injectable silk fibroin-based composition can be present in any state that can be administered to a tissue by injection. Thus, an injectable silk fibroin-based composition can be in a form of a liquid, a colloid, a gel, a paste, a viscous solution, a flowable material, or any combinations thereof. The term “flowable material” describes any injectable material that flows as a uniform or homogenous mass when an appropriate pressure is applied. Such flowable materials can include, but are but not limited to, liquids (including viscous liquids), solutions (including viscous solutions), emulsions, colloids, suspensions, slurries, pastes, gels, or any combinations thereof. In some embodiments, an injectable silk fibroin-based composition is a liquid or solution, e.g., where silk fibroin, and optionally functionally-activated PEG components are prepared in a solution (or a viscous solution). In some embodiments, an injectable silk fibroin-based composition can be a colloid or suspension, e.g., a plurality of silk fibroin-based particles dispersed in a liquid or solution. In such embodiments, silk fibroin-based particles can be produced, e.g., by forming a silk fibroin-based gel from a silk fibroin-based composition using any methods described herein or recognized in the art, followed by reducing the gel into particles, particulates or powder. The silk fibroin-based gel particles, particulates or powder can swell upon injection into a tissue (e.g., a cervical tissue) to enhance mechanical stiffness of the tissue (e.g., a cervical tissue).

### Exemplary Active Agents

In some embodiments, a silk fibroin-based composition can further comprise at least one active agent. Examples of active agents can be selected from the group consisting of cells, proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, prodrugs, therapeutic agents (e.g., chemotherapeutic agents), small molecules, drugs, and any combinations thereof.

In particular embodiments, the active agent can include a population of cervical cells, for example, about 10³ cervical cells, about 10⁴ cervical cells, about 10⁵ cervical cells, about 10⁶ cervical cells, about 10⁷ cervical cells or more. The term “cervical cells” as used herein, generally refers to squamous epithelial cells and/or glandular cells that line the surface of the cervix. In some embodiments, the cervical cells include cervical fibroblasts. The term “cervical cells” as used herein can include normal cervical cells, as well as genetically modified cervical cells, or can be derived from stem cells. The cervical cells used in the method described herein can be autologous or allogeneic. Cervical cells can be collected from a cervix of a patient (a subject or an individual, preferably a female), for example, by cervical or other cell surface scrape, or needle biopsy or tissue biopsy. In some embodiments, the collected cervical cells can be cultured in vitro to expand the population prior to injection. In some embodiments, the cervical cells used in the methods described herein can be preserved in a liquid medium and stored for a period of time prior to use. In some embodiments, the cervical cells used in the methods described herein can be present in a cell nutrient medium. In some embodiments, the cervical cells can include cervical fibroblasts.

Other cell types can be used when the methods described herein are used for treatment of other incompetent tissues. Exemplary cells suitable for use herein can include, but are not limited to, progenitor cells or stem cells, smooth muscle cells, skeletal muscle cells, cardiac muscle cells, epithelial cells, endothelial cells, urothelial cells, fibroblasts, myoblasts, oscular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, and precursor cells. The active agents can also be the combinations of any of the cells listed above. See also WO 2008/106485; PCT/US2009/059547; and WO 2007/103442.

In some embodiments, the active agent can include a matricellular glycoprotein, e.g., thrombospondin 2.

In some embodiments, the active agent can include a serine protease protein, e.g., thrombin.

In some embodiments, the active agent can include a fibrillar protein that can polymerize to form a network structure. An exemplary fibrillar protein can include fibrin. Fibrin can be produced by conversion of fibrinogen by enzymatic reactions using thrombin. Accordingly, in some embodiments, the active agent can include fibrinogen and thrombin.

In other embodiments, the active agent can include a chemical agent for various properties and applications. In some embodiments, the chemical agent can be an adhesive such as cyanoacrylate-based adhesives. Cyanoacrylate is the generic name for cyanoacrylate-based adhesives such as methyl 2-cyanoacrylate, ethyl-2-cyanoacrylate, n-butyl cyanoacrylate, 2-octyl cyanoacrylate, and any derivatives thereof. In certain embodiments, the cyanoacrylate can include a medical grade adhesive, e.g., 2-octyl cyanoacrylate under various trade names such as derma+flex QS, SurgiSeal, octylseal, FloraSeal, Dermabond, Surgi-Lock and Nexaband. Additional cyanoacrylate-based adhesives that can be used in the methods described herein include, but are not limited to, the ones described in U.S. Pat. App. Nos. US 2006/0251612; US 2008/0241249; US 2008/0003196; and U.S. Pat. Nos. 6,224,622 and 7,351,426. In some embodiments, the cyanoacrylate can include n-butyl cyanoacrylate (e.g., used in the veterinary glues Vetbond and LiquiVet and skin glues like GluStitch, Xoin, Indermil, LiquiBand and Histoacryl).

In other embodiments, the chemical agent can be a crosslinking agent. In certain embodiments, the chemical agent can be a protein-crosslinking agent. An exemplary crosslinking agent includes, but not limited to, glutaraldehyde and any art-recognized crosslinking agents that are biocompatible.

In various embodiments, the active agent can include an extracellular matrix protein. As used herein, the term “extracellular matrix protein” generally refers to a protein which constitutes an extracellular matrix. Examples of an extracellular matrix protein that can be included in the silk fibroin-based compositions and/or in the methods described herein include, but are not limited to, osteopontin, vitronectin, fibronectin, von Willebrand Factor, collagen, laminin, tenascin, fibrinogen, thrombospondin, angiostatin, plasmin, proteoglycans, hyaluronan, elastin and VCAM-1. As used herein, the term “extracellular matrix” refers to a complex aggregate of biological polymers, which fills the extracellular space in tissue, e.g., a cervical tissue, in accordance with the meaning commonly used in the art (for example, see “Dictionary of Molecular Cell Biology”, page 323, Tokyo Kagaku Dozin Co., Ltd., 1997). In some embodiments, the extracellular matrix protein can be any art-recognized extracellular matrix protein present in the cervix (See, e.g., House M. et al. 2009. Semin Perinatol. 33(5): 300). Examples of such extracellular matrix protein found in a cervix or a cervical tissue can include, but are not limited to, collagen, proteoglycans, hyaluronan, elastin, and any combinations thereof. In one embodiment, the active agent can include collagen.

### Subjects Amenable to the Methods Described Herein

The methods described herein can be used to treat any subject in need thereof. In some embodiments, the methods described herein can further comprising selecting subjects in need thereof.

In some embodiments, the subject in need thereof can be at risk of, or diagnosed with cervical insufficiency. Methods for diagnosing cervical insufficiency are well known to a skilled practitioner, including, but not limited to, measuring the cervical opening and/or cervical length using transvaginal ultrasound, MRI, CT, or ultrasonography. Generally, a subject with a cervical length of less than 25 mm and/or a cervical opening of greater than 10 mm can be diagnosed with or at risk of cervical insufficiency. In some embodiments, a subject with a cervical length of less than 25 mm in the midtrimester can be diagnosed with or at risk of cervical insufficiency. Alternatively, methods for measuring mechanical stiffness of a cervical tissue in vivo, as described earlier, can be used for diagnosis of a “weakened,” “incompetent,” or “softened” cervical tissue, i.e. cervix dilation without uterine contractility, which can in turn result in miscarriage or preterm birth. Such subjects with a “weakened,” “incompetent” or “softened” cervical tissue can be also amenable to the methods described herein.

Patients previously diagnosed with known cervical insufficiency and an obstetric history significant for repeated pregnancy loss between about 10-30 weeks or about 16-24 weeks can also be treated with the methods described herein.

Other risk factors for cervical insufficiency can include, but are not limited to, diagnosis of cervical incompetence in a previous pregnancy, previous preterm premature rupture of membranes, history of conization (cervical biopsy), diethylstilbestrol exposure, which can cause anatomical defects, uterine anomalies, and any combinations thereof. Repeated procedures (such as mechanical dilation, especially dining late pregnancy) can also create a risk for cervical insufficiency. Additionally, any significant trauma to a cervix can weaken the tissues involved and can potentially lead to an incompetent cervical tissue. One of skill in the art can make such diagnosis with a review of the obstetric history,

In some embodiments, the subject amenable to the methods described herein can have a history of preterm birth. The term “preterm birth” as used herein refers to the birth of a human baby of less than 37 weeks gestational age. Premature birth, commonly used as a synonym for preterm birth, can also refer to the birth of a baby before the developing organs are mature enough to allow normal postnatal survival. In some embodiments, the subject amenable to the methods described herein can have a history of preterm birth and a cervical length of less than 25 mm.

In some embodiments, the subjects diagnosed with fetal membranes (or amniotic membranes) protruding through the external os before fetal viability can be treated with the methods described herein. One of skill in the art can make such diagnosis in a physical examination.

In some embodiments, the subjects in need thereof are the ones who are prescribed bedrest to reduce cervical loading gravity.

In some embodiments, the subjects in need thereof can be the ones with a dilated cervix in the midtrimester during pregnancy.

In some embodiments, the subject in need thereof can be a subject that is recommended to receive or has received a treatment comprising a cervical cerclage. In these embodiments, the subject recommended to receive a treatment comprising a cervical cerclage can be selected for administration of one or more embodiments of the method of treatment described herein without a treatment comprising a cervical cerclage. In other embodiments, the subject recommended to receive a cervical cerclage can be selected for administration of one or more embodiments of the method of treatment described herein in conjunction with a treatment comprising a cervical cerclage. In some embodiments, the subjects amenable to the methods described herein can have already had at least one cervical cerclage in place. In some embodiments, the methods described herein can be used in place of a cervical cerclage.

In some embodiments, the subject amenable to the methods described herein can be at risk of, or diagnosed with a multiple gestation. Multiple gestation, or multiple pregnancy, can occur when two or more fetuses are conceived at the same time in the same woman. However, multiple gestation may or may not result in the live births of multiple babies. In some cases, the woman's body can naturally reduce the number of fetuses present, or a woman can decide to reduce the number due to the health risks associated with multiple gestation.

With multiple gestation, the fetuses are either monozygotic or dizygotic. Monozygotic means that, during conception, the sperm fertilizes one egg, which will later split into two or more developing embryos. These types of siblings are genetically identical and almost always the same sex. Dizygotic multiples occur when a woman's body releases several eggs, and those eggs are each fertilized by different sperm. The resulting fetuses are fraternal siblings, and are not identical. Fraternal twins or triplets are typically only as similar to one another as other regular siblings.

In some embodiments, the subject amenable to the methods described herein can be a subject prescribed with progesterone therapy. In such embodiments, the method described herein can be used in conjunction with a treatment comprising progesterone administration. In some embodiments, the subjects amenable to the methods described herein can have already administered at least once with progesterone. In some embodiments, the method described herein can be used in place of progesterone therapy.

In some embodiments, a subject with an incompetent or dilated tissue other than a cervical tissue can also be treated with the methods described herein. Exemplary examples of incompetent tissues (e.g., “weakened” tissue or “softened” tissue) or dilated tissue structures include, but are not limited to, valve repair, gastro-esophageal sphincter, bladder sphincter, bladder neck or an anal sphincter.

As used herein, a “subject” generally means a human or animal. In some embodiments, the subjects are females. In some embodiments, the subjects are pregnant subjects. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. In addition, the methods and compositions described herein can be used to treat domesticated animals and/or pets.

### Exemplary Delivery Devices of a Silk Fibroin-Based Composition

In some embodiments, a silk fibroin-based composition is administered to a cervical tissue by injection. Accordingly, pre-loaded delivery devices for injection of a silk fibroin-based material to a cervical tissue in treatment of cervical insufficiency are also provided herein. For example, as shown in FIG. 1 for illustration purposes only, a pre-loaded delivery device 100 comprises (i) a first chamber 102 containing a first functionally activated PEG component; and further comprising a first port at a first end 104 for passage of the first functionally activated PEG component and a first movable plunger 106 disposed at a second end; (ii) a second chamber 108 containing a second functionally activated PEG component capable of reacting with the first functionally activated PEG component to form a crosslinked matrix; and further comprising a first port at a first end 110 for passage of the second functionally activated PEG component and a second movable plunger 112 disposed at a second end; wherein at least one of the first chamber 102 and the second chamber 108 further comprises silk fibroin at a concentration of at least about 5 wt % capable of forming beta-sheets to further stabilize the crosslinked matrix.

In some embodiments, the first functionally activated PEG component can comprise at least one maleimidyl group.

In some embodiments, the second functionally activated PEG component can comprise at least one thiol group.

In some embodiments, the first movable plunger 106 and the second movable plunger 112 can be mechanically connected to each other such that motion of one movable plunger in one direction is configured to have another movable plunger to move in the same direction.

In some embodiments, the delivery device can comprise a double-barrel syringe, e.g., but not limited to, the devices described in U.S. Pat. Nos. 6,065,645; 6,732,887; 7,530,808; 8,052,421; and U.S. Pat. App. No. US 2009/0152300, or any modifications to art-recognized double-barrel syringes that are known to one of skill in the art.

In some embodiments, the delivery device can further comprise a mixing tip 114. A mixing tip 114 can be of any shape, e.g., a conical shape, or a cylindrical shape. A mixing tip 114 generally comprises a bore connecting between an inlet and an exit end, e.g., for discharge for a flowable material, where a mixing structural element (e.g., a static or spiral mixing element) 116 is disposed inside the bore of the mixing tip 114. The inlet of the mixing tip 114 can be adaptably engaged to both the first ports of the first chamber and the second chamber 104, 110 such that the first functionally-activated PEG component in the first chamber 102 and the second functionally-activated PEG component in the second chamber 108 can be mixed together while flowing through the mixing tip 114. In addition, the mixing tip 114 can be detachable after it is engaged to the delivery device, so that the mixing tip 114 can be replaced with a locking cap, if needed. Any art-recognized mixing tip commonly used with a syringe, e.g., but not limited to the ones described in U.S. Pat. No. 6,564,972 can be used with any embodiments of the delivery devices described herein.

In some embodiments, the delivery device can further comprise a needle, e.g., to facilitate delivery of the components inside the chambers to a tissue (e.g., a cervical tissue). In some embodiments, the needle can be adaptably engaged to the mixing tip 114 such that the mixed components exiting from the mixing tip 114 can then be subsequently injected into a tissue (e.g., a cervical tissue) through the needle. In other embodiments, the exit end of the mixing tip 114 can be configured to shape like a needle. The needle can have a gauge of any size suitable to inject a composition into a tissue (e.g., a cervical tissue). In some embodiments, the needle can have a gauge of at least about 15, or at least about 20.

In some embodiments, when the delivery device is not in use, the delivery device can further comprise at least one cap (e.g., a locking cap) to seal, directly or indirectly, the first ports of the first chamber and the second chamber 104, 110, e.g., for preventing each individual PEG components from flowing out, and/or from contamination. For example, the cap can be used to seal the exit end of the mixing tip 114, or it can be used to directly seal the first ports of the first chamber and the second chamber 104, 110, e.g., after removal of the mixing tip 114.

While silk fibroin can be blended with at least one of the first functionally-activated PEG component and the second functionally-activated PEG component in the corresponding chambers, silk fibroin can also be separated from the first functionally-activated PEG component and the second functionally-activated PEG component and be contained in a third chamber further comprising a first port at a first end for passage of the silk fibroin and a third movable plunger disposed at a second end. In this embodiment, silk fibroin can be mixed together with the first functionally-activated PEG component and the second functionally-activated PEG component when all three components are flowing through a mixing tip.

### Kits

Kits for treatment of an incompetent cervix and/or cervical insufficiency are also provided herein. In some embodiments, a kit can comprise a delivery device containing a silk fibroin-based composition that forms a gel upon injection into a cervix, and a speculum.

In some embodiments, the silk fibroin-based composition as described herein can comprise at least three components including silk fibroin and at least two functionally activated PEG components. Different embodiments of silk fibroin and PEG components of the composition are described earlier. In some embodiments, the silk fibroin-based compositions can be pre-loaded into a delivery device, e.g., a double-barreled injection device. In such embodiments, by way of example only, one PEG component mixed with silk fibroin can be pre-loaded in one barrel of the delivery device, while another PEG component optionally mixed with silk fibroin can be pre-loaded in another barrel of the delivery device. The components inside the barrels can be present in powder, which will be suspended into a solution at time of use, or they can be pre-suspended in a solution. In other embodiments, the components of the silk fibroin-based compositions can be pre-loaded into separate delivery devices, e.g., syringes.

In some embodiments, a kit can comprise (i) one or more delivery devices described herein, e.g., a pre-loaded delivery device 100, (ii) at least one mixing tip 114, and at least one reagent. Examples of reagents that can be included in a kit can include, but are not limited to, a solvent for at least one of the first functionally activated PEG component, the second functionally activated PEG component and silk fibroin; a beta sheet-inducing agent; or a combination thereof. In some embodiments, the solvent can include an aqueous solution, e.g., water. A “beta-sheet inducing agent” is an agent that can induce beta-sheet formation in silk fibroin, and can include, without limitations, an alcohol solution, an acid solution, or a combination thereof.

In some embodiments, the kit can further comprise at least one needle. The needle can be adaptably engaged to a mixing tip provided in the kit.

In some embodiments, the kit can further comprise a disposable speculum.

In some embodiments of any aspects of kits described herein, the kit can further comprise an active agent described herein. The active agent can be provided in a separate container or package, or mixed with a silk fibroin-based composition or at least one component contained in the delivery device.

Some embodiments of the kit can further comprise instructions, e.g., describing how the contents of the kit are used to carry out one or more methods described herein. Instructions can include steps and conditions necessary to inject the silk fibroin-based composition into a cervix. Instructions supplied in the kits can include written instructions on a label or package insert (e.g., a paper sheet included in the kit), or machine-readable instructions (e.g., instructions carried on a magnetic or optical storage disk).

Embodiments of the various aspects described herein can be illustrated by the following numbered paragraphs.
1. A method for increasing mechanical stiffness of a cervical tissue in a subject, comprising placing a silk fibroin-based composition into at least a portion of a cervix of the subject, wherein the silk fibroin-based composition forms a gel upon placement into the cervix, thereby increasing the mechanical stiffness of the cervical tissue in the subject.
2. The method of paragraph 1, wherein the placing comprises injecting the silk fibroin-based composition into said at least a portion of the cervix of the subject.
3. The method of paragraph 1 or 2, wherein the at least a portion of the cervix is a stroma of the cervix.
4. The method of any of paragraphs 1-3, wherein the silk fibroin-based composition comprises silk fibroin having a concentration of about 5 wt % to about 30 wt %.
5. The method of paragraph 4, wherein the silk fibroin has a concentration of about 5 wt % to about 10 wt %.
6. The method of any of paragraphs 1-5, wherein the silk fibroin-based composition comprises at least two functionally-activated PEG components capable of reacting with one another to form a crosslinked matrix, and the silk fibroin capable of forming beta-sheets to further stabilize the crosslinked matrix.
7. The method of paragraph 6, further comprising mixing the PEG components and the silk fibroin.
8. The method of any of paragraphs 6-7, wherein each PEG component is a four-armed PEG.
9. The method of any of paragraphs 6-8, wherein one of the PEG components is functionally activated with a maleimidyl group.
10. The method of any of paragraphs 6-9, wherein one of the PEG components is functionally activated with a thiol group.
11. The method of any of paragraphs 6-10, wherein one of the PEG components is functionally activated with a maleimidyl group and another one of the PEG components is functionally activated with a thiol group.
12. The method of any of paragraphs 1-11, further comprising exposing the silk fibroin-based composition, upon the placement into the cervix, to a treatment comprising ultrasound for a sufficient period of time to initiate gelation.
13. The method of any of paragraphs 1-12, further comprising exposing the silk fibroin-based composition, upon the placement into the cervix, to a treatment comprising an electric field for a sufficient period of time to initiate gelation.
14. The method of any of paragraphs 1-13, further comprising exposing the silk fibroin-based composition, upon the placement into the cervix, to an alcohol treatment or a water-annealing treatment.
15. The method of any of paragraphs 1-14, further comprising exposing the cervix with a speculum prior to the placement of the silk fibroin-based composition into the cervix of the subject.
16. The method of any of paragraphs 1-15, wherein the subject is at risk of, or diagnosed with cervical insufficiency.
17. The method of any of paragraphs 1-16, wherein the subject has a history of preterm birth.
18. The method of paragraph 17, wherein the subject has a cervical length of less than 2.5 cm.
19. The method of any of paragraphs 1-18, wherein the subject is indicated to a treatment comprising a cervical cerclage.
20. The method of any of paragraphs 1-19, wherein the subject is at risk of, or diagnosed with a multiple gestation.
21. The method of any of paragraphs 1-20, wherein the method is used in conjunction with a treatment comprising a cervical cerclage.
22. The method of any of paragraphs 1-21, wherein the silk fibroin-based composition further comprises an active agent.
23. The method of paragraph 22, wherein the active agent is selected from the group consisting of cells, proteins, peptides, nucleic acids, aptamers, antibodies or fragments or portions thereof, antigens, hormones, hormone antagonists, growth factors or recombinant growth factors and fragments and variants thereof, cell attachment mediators, enzymes, antibiotics, viruses, prodrugs, therapeutic agents, small molecules, and any combinations thereof.
24. The method of any of paragraphs 22-23, wherein the active agent is a population of cervical cells.
25. The method of any of paragraphs 22-23, wherein the active agent is selected from a group consisting of thrombospondin 2, thrombin, fibrin, cyanoacrylate, glutaraldehyde, and any combinations thereof.
26. The method of any of paragraphs 1-25, wherein the silk fibroin-based composition further comprises an extracellular matrix selected from a group consisting of collagen, proteoglycans, hyaluronan, elastin, and any combinations thereof.
27. The method of any of paragraphs 1-26, wherein the silk fibroin-based composition further comprises collagen.
28. The method of any of paragraphs 1-27, wherein the method is used in conjunction with progesterone therapy.
29. Use of a silk fibroin-based composition for increasing mechanical stiffness of a cervical tissue of a subject, wherein the silk fibroin-based composition forms a gel upon placement into the cervical tissue of the subject.
30. Use of a silk fibroin-based composition for treatment of cervical insufficiency in a subject, wherein the silk fibroin-based composition forms a gel upon placement into a cervical tissue of the subject.
31. The use of paragraph 29 or 30, wherein the silk fibroin-based composition is injectable.
32. The use of any of paragraphs 29-31, wherein the silk fibroin-based composition comprises silk fibroin having a concentration of about 5 wt % to about 30 wt %.
33. The use of any of paragraphs 29-32, wherein the silk fibroin has a concentration of about 5 wt % to about 10 wt %.
34. The use of any of paragraphs 29-33, wherein the silk fibroin-based composition comprises at least two functionally-activated PEG components capable of reacting with one another to form a crosslinked matrix, and the silk fibroin capable of forming beta-sheets to further stabilize the crosslinked matrix.
35. The use of any of paragraphs 29-34, wherein each PEG component is a four-armed PEG.
36. The use of any of paragraphs 29-35, wherein one of the PEG components is functionally activated with a maleimidyl group.
37. The use of any of paragraphs 29-36, wherein one of the PEG components is functionally activated with a thiol group.
38. The use of any of paragraphs 29-37, wherein one of the PEG components is functionally activated with a maleimidyl group and another one of the PEG components is functionally activated with a thiol group.
39. The use of any of paragraphs 29-38, wherein the silk fibroin-based composition, upon the placement into the cervix, is further exposed to a treatment comprising ultrasound for a sufficient period of time to initiate gelation.
40. The use of any of paragraphs 29-39, wherein the silk fibroin-based composition, upon the placement into the cervix, is further exposed to a treatment comprising an electric field for a sufficient period of time to initiate gelation.
41. The use of any of paragraphs 29-40, wherein the silk fibroin-based composition, upon the placement into the cervix, is further exposed to an alcohol treatment or a water-annealing treatment.
42. The use of any of paragraphs 29-41, wherein the subject is at risk of, or diagnosed with cervical insufficiency.
43. The use of any of paragraphs 29-42, wherein the subject has a history of preterm birth.
44. The use of paragraph 43, wherein the subject has a cervical length of less than 2.5 cm.
45. The use of any of paragraphs 29-44, wherein the subject is indicated to a treatment comprising a cervical cerclage.
46. The use of any of paragraphs 29-45, wherein the subject is at risk of, or diagnosed with a multiple gestation.
47. The use of any of paragraphs 29-46, wherein the silk fibroin-based composition is used in conjunction with a treatment comprising a cervical cerclage.
48. The use of any of paragraphs 29-47, wherein the silk fibroin-based composition further comprises an active agent.
49. The use of paragraph 48, wherein the active agent is selected from the group consisting of cells, proteins, peptides, nucleic acids, aptamers, antibodies or fragments or portions thereof, antigens, hormones, hormone antagonists, growth factors or recombinant growth factors and fragments and variants thereof, cell attachment mediators, enzymes, antibiotics, viruses, prodrugs, therapeutic agents, small molecules, and any combinations thereof.
50. The use of any of paragraphs 48-49, wherein the active agent is a population of cervical cells.
51. The use of any of paragraphs 48-49, wherein the active agent is selected from a group consisting of thrombospondin 2, thrombin, fibrin, cyanoacrylate, glutaraldehyde, and any combinations thereof.
52. The use of any of paragraphs 29-51, wherein the silk fibroin-based composition further comprises an extracellular matrix selected from a group consisting of collagen, proteoglycans, hyaluronan, elastin, and any combinations thereof.
53. The use of any of paragraphs 29-52, wherein the silk fibroin-based composition further comprises collagen.
54. The use of any of paragraphs 29-53, wherein the method is used in conjunction with progesterone therapy.
55. A pre-loaded delivery device for use in treatment of cervical insufficiency comprising
   a first chamber containing a first functionally activated PEG component; and further comprising a first port at a first end for passage of the first functionally activated PEG component and a first movable plunger disposed at a second end;
   a second chamber containing a second functionally activated PEG component capable of reacting with the first functionally activated PEG component to form a crosslinked matrix; and further comprising a first port at a first end for passage of the second functionally activated PEG component and a second movable plunger disposed at a second end;
   wherein at least one of the first and the second chambers further comprises silk fibroin at a concentration of at least about 5 wt % capable of forming beta-sheets to further stabilize the crosslinked matrix.
56. The delivery device of paragraph 55, wherein the delivery device comprises a double-barrel syringe.
57. The delivery device of paragraph 55 or 56, further comprising a cap to seal the first ports of the first chamber and the second chamber when the delivery device is not in use.
58. The delivery device of any of paragraphs 55-57, further comprising a mixing tip simultaneously engageable to both the first ports of the first chamber and the second chamber.
59. The delivery device of paragraph 58, further comprising a needle engageable to the mixing tip.
60. The delivery device of any of paragraph 55-58, further comprising a needle simultaneously engageable to both the first ports of the first chamber and the second chamber.
61. The delivery device of any of paragraphs 59-60, wherein the needle has a gauge of at least about 15.
62. The delivery device of any of paragraphs 55-61, wherein the first functionally activated PEG component comprises at least one maleimidyl group.
63. The delivery device of any of paragraphs 55-62, wherein the second functionally activated PEG component comprises at least one thiol group.
64. A kit for use in treatment of cervical insufficiency comprising:
   at least one pre-loaded delivery device comprising:
      a first chamber containing a first functionally activated PEG component; and further comprising a first port at a first end for passage of the first functionally activated PEG component and a first movable plunger disposed at a second end;
      a second chamber containing a second functionally activated PEG component capable of reacting with the first functionally activated PEG component to form a crosslinked matrix; and further comprising a first port at a first end for passage of the second functionally activated PEG component and a second movable plunger disposed at a second end;
         wherein at least one of the first and the second chambers further comprises silk fibroin at a concentration of at least about 5 wt % capable of forming beta-sheets to further stabilize the crosslinked matrix;
   at least one mixing tip simultaneously engageable to the first ports of both the first chamber and the second chamber; and
   at least one reagent.
65. The kit of paragraph 64, further comprising at least one needle simultaneously engageable to said at least one mixing tip.
66. The kit of paragraph 64 or 65, further comprising a disposable speculum.
67. The kit of any of paragraphs 64-66, wherein said at least one reagent includes a solvent for at least one of the first functionally activated PEG component, the second functionally activated PEG component and silk fibroin; a beta sheet-inducing agent; or a combination thereof.
68. The kit of any of paragraphs 67, wherein the solvent includes an aqueous solution.
69. The kit of paragraph 68, wherein the aqueous solution is deionized water.
70. The kit of any of paragraphs 64-69, wherein the beta-sheet inducing agent includes an alcohol solution, an acid solution, or a combination thereof.

### DEFINITIONS OF SOME SELECTED TERMS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not. Additionally, the term “comprising” or “comprises” includes “consisting essentially of” and “consisting of.”

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages can mean ±1%.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The term “statistically significant” or “significantly” refers to statistical significance and generally means a two standard deviation (2SD) above or below a reference level. The term refers to statistical evidence that there is a difference. It is defined as the probability of making a decision to reject the null hypothesis when the null hypothesis is actually true. The decision is often made using the p-value.

As used herein, the term “substantially” means a proportion of at least about 60%, or preferably at least about 70% or at least about 80%, or at least about 90%, at least about 95%, at least about 97% or at least about 99% or more, or any integer between 70% and 100%. In some embodiments, the term “substantially” means a proportion of at least about 90%, at least about 95%, at least about 98%, at least about 99% or more, or any integer between 90% and 100%. In some embodiments, the term “substantially” can include 100%.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

The term “nucleic acids” used herein refers to polymers (polynucleotides) or oligomers (oligonucleotides) of nucleotide or nucleoside monomers consisting of naturally occurring bases, sugars and intersugar linkages. The term “nucleic acid” also includes polymers or oligomers comprising non-naturally occurring monomers, or portions thereof, which function similarly. Exemplary nucleic acids include, but are not limited to, deoxyribonucleic acid (DNA), ribonucleic acid (RNA), locked nucleic acid (LNA), peptide nucleic acids (PNA), and polymers thereof in either single- or double-stranded form. Locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo conformation. LNA nucleotides can be mixed with DNA or RNA residues in the oligonucleotide whenever desired Such LNA oligomers are generally synthesized chemically. Peptide nucleic acid (PNA) is an artificially synthesized polymer similar to DNA or RNA. DNA and RNA have a deoxyribose and ribose sugar backbone, respectively, whereas PNA's backbone is composed of repeating N-(2-aminoethyl)-glycine units linked by peptide bonds. PNA is generally synthesized chemically. Unless specifically limited, the term “nucleic acids” encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term “nucleic acid” should also be understood to include, as equivalents, derivatives, variants and analogs of either RNA or DNA made from nucleotide analogs, and, single (sense or antisense) and double-stranded polynucleotides.

The term “enzymes” as used here refers to a protein molecule that catalyzes chemical reactions of other substances without it being destroyed or substantially altered upon completion of the reactions. The term can include naturally occurring enzymes and bioengineered enzymes or mixtures thereof. Examples of enzyme families include kinases, dehydrogenases, oxidoreductases, GTPases, carboxyl transferases, acyl transferases, decarboxylases, transaminases, racemases, methyl transferases, formyl transferases, and α-ketodecarboxylases.

As used herein, the term “aptamers” means a single-stranded, partially single-stranded, partially double-stranded or double-stranded nucleotide sequence capable of specifically recognizing a selected non-oligonucleotide molecule or group of molecules. In some embodiments, the aptamer recognizes the non-oligonucleotide molecule or group of molecules by a mechanism other than Watson-Crick base pairing or triplex formation. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. Methods for selecting aptamers for binding to a molecule are widely known in the art and easily accessible to one of ordinary skill in the art.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments can be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (113) in the heavy chain variable domain; Chothia and Lesk J. Mol. Biol. 196:901-917 (1987)). In some instances, a complementarity determining region can include amino acids from both a CDR region defined according to Kabat and a hypervariable loop.

The expression “linear antibodies” refers to the antibodies described in Zapata et al., Protein Eng., 8(10):1057-1062 (1995). Briefly, these antibodies comprise a pair of tandem Fd segments (VH -CH1-VH-CH1) which, together with complementary light chain polypeptides, form a pair of antigen binding regions. Linear antibodies can be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (Plückthun, The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, P0:6444-6448 (1993)).

As used herein, the term “small molecules” refers to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “cells” used herein generally refers to any cell, prokaryotic or eukaryotic, including plant, yeast, worm, insect and mammalian. Mammalian cells include, without limitation; primate, human and a cell from any animal of interest, including without limitation; mouse, hamster, rabbit, dog, cat, domestic animals, such as equine, bovine, murine, ovine, canine, feline, etc.

As used herein, the term “antigens” refers to a molecule or a portion of a molecule capable of being bound by a selective binding agent, such as an antibody, and additionally capable of being used in an animal to elicit the production of antibodies capable of binding to an epitope of that antigen. An antigen may have one or more epitopes. The term “antigen” can also refer to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

The term “antibiotics” as used herein can encompass any pharmaceutically acceptable compound that can inhibit the growth of or destroy bacteria and/or other microbes, regardless of whether the compound is produced in a microorganism or produced synthetically. The term “antibiotics” can encompass disinfectants, antiseptics, and any other antimicrobial compounds. For example, the term “antibiotic” can encompass penicillin and all its derivatives. Exemplary antibiotics include, but are not limited to, actinomycin; aminoglycosides (e.g., neomycin, gentamicin, tobramycin); β-lactamase inhibitors (e.g., clavulanic acid, sulbactam); glycopeptides (e.g., vancomycin, teicoplanin, polymixin); ansamycins; bacitracin; carbacephem; carbapenems; cephalosporins (e.g., cefazolin, cefaclor, cefditoren, ceftobiprole, cefuroxime, cefotaxime, cefipeme, cefadroxil, cefoxitin, cefprozil, cefdinir); gramicidin; isoniazid; linezolid; macrolides (e.g., erythromycin, clarithromycin, azithromycin); mupirocin; penicillins (e.g., amoxicillin, ampicillin, cloxacillin, dicloxacillin, flucloxacillin, oxacillin, piperacillin); oxolinic acid; polypeptides (e.g., bacitracin, polymyxin B); quinolones (e.g., ciprofloxacin, nalidixic acid, enoxacin, gatifloxacin, levaquin, ofloxacin, etc.); sulfonamides (e.g., sulfasalazine, trimethoprim, trimethoprim-sulfamethoxazole (co-trimoxazole), sulfadiazine); tetracyclines (e.g., doxycyline, minocycline, tetracycline, etc.); monobactams such as aztreonam; chloramphenicol; lincomycin; clindamycin; ethambutol; mupirocin; metronidazole; pefloxacin; pyrazinamide; thiamphenicol; rifampicin; thiamphenicl; dapsone; clofazimine; quinupristin; metronidazole; linezolid; isoniazid; piracil; novobiocin; trimethoprim; fosfomycin; fusidic acid; or other topical antibiotics. Optionally, the antibiotic agents can also be antimicrobial peptides such as defensins, magainin and nisin; or lytic bacteriophage. The antibiotic agents can also be the combinations of any of the agents listed above. See also PCT/US2010/026190.

The term “therapeutic agents” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject. Examples include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, proteins, antibodies, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals.

As used herein, the term “hormones” generally refers to naturally or non-naturally occurring hormones, analogues and mimics thereof. In certain embodiments, the term “hormones” refers to any hormones used in therapeutic treatment, e.g., growth hormone treatment. As used herein, “growth hormone” or “GH” refers to growth hormone in native-sequence or in variant form, and from any source, whether natural, synthetic, or recombinant. Examples include human growth hormone (hGH), which is natural or recombinant GH with the human native sequence (somatotropin or somatropin), and recombinant growth hormone (rGH), which refers to any GH or variant produced by means of recombinant DNA technology, including somatrem, somatotropin, and somatropin. In one embodiment, hormones include insulin.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1: A Silk Fibroin-Based Injectable Biomaterial as an Alternative to Cervical Cerclage

Spontaneous preterm birth is a significant public health problem, complicating over 12% of pregnancies in the United States (1) and resulting in $26 billion in health care costs (2). Preterm birth before 32 weeks is the most frequent cause of neonatal mortality (3) Preterm birth is also a common cause of long-term neurodevelopmental disability and cerebral palsy (4). While preterm birth is a complex, multifactorial disorder, a dysfunctional cervix is associated with a significant number of preterm deliveries. Cervical shortening is an important risk factor for preterm birth (5, 6) and a short cervix is generally used to screen patients for therapy to prevent preterm birth (7, 8). There are two common treatment alternatives when cervical dysfunction is suspected: progesterone supplementation and cervical cerclage (9). However, these treatments are not effective for many patients and there is a need for new treatment alternatives to improve care for women at risk for preterm birth.

In many cases, the pathophysiology of cervical dysfunction is related to weakened biomechanical properties of the cervical stroma (10). The cervical stroma is composed of fibrous connective tissue that undergoes complex remodeling in preparation for labor (11). A cerclage therapy is usually performed to prevent preterm shortening and insufficiency by providing support to a weakened cervical tissue. Although placement of a cerclage is effective in some patients (12), cerclage failure is not uncommon. These failures may occur because a cerclage does not prevent excessive tissue softening, which is the primary pathophysiology of cervical dysfunction. A novel therapy that enhances the biomechanical properties of a cervix, e.g., by increasing its tissue stiffness, can be more effective than cerclage therapy.

A two-part, silk-polyethylene glycol (PEG) biomaterial for use as a tissue sealant was previously reported (See, e.g., Serban et al. J Biomed Mater. Res A 2011; 98:567-575; and WO 2012/031144, the contents of which are incorporated herein by reference). Without wishing to be bound by theory, the silk-PEG biomaterial rapidly gelled after mixing because of covalent crosslinks formed between chemically active functional groups on the PEG. In addition, the silk-PEG biomaterial was induced to form beta sheets from the silk components to enhance mechanical properties. The silk-PEG biomaterial showed improved mechanical properties and decreased swelling compared to a commercially available PEG sealant (21). However, it is not known whether an injectable, silk-PEG biomaterial can be used to increase the mechanical stiffness of a cervical tissue, e.g., to provide load-bearing support during pregnancy.

Accordingly, in order to investigate the ability of an injectable silk-PEG biomaterial to increase the mechanical stiffness of a cervical tissue, the biomaterial was injected into a human cervical tissue and evaluated for mechanical properties, gelation, swelling, and cytocompatibility.

### Exemplary Methods and Materials

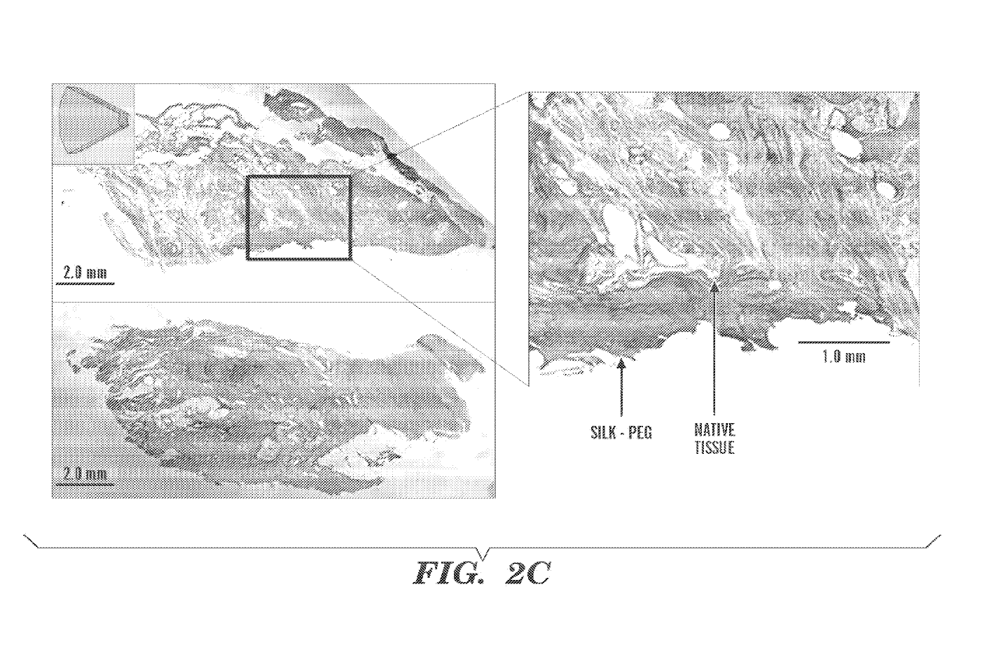

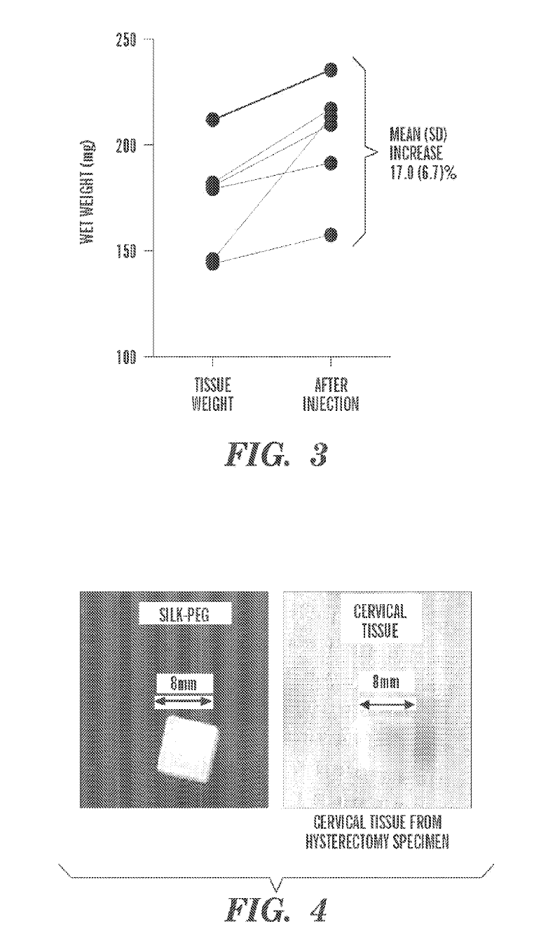

Cervical tissues: Human cervical tissue specimens were obtained from premenopausal gynecological hysterectomies for benign indications (N=8). Informed consent was obtained. Immediately after removal of the uterus and cervix, a circumferential portion of the cervix between the internal and external os was removed and placed in ice cold saline (FIG. 2A). The specimens were used fresh or snap frozen in liquid nitrogen and stored at −80° C.

Silk fibroin extraction: Silk fibroin protein was purified in accordance to a method known in the art (See, e.g., Ref. 22). For example, Bombyx mori cocoons (Tajima Shoji Co., LTD, Japan) were cut into about 1 cm pieces and boiled in an aqueous 0.2 M sodium carbonate solution for about 30 minutes. The fibrous silk was solubilized in about 9.3 M lithium bromide at ˜60° C. for about 4 hours and dialyzed against distilled water for about 72 hours to obtain a ˜6% (w/v) silk solution. To increase the concentration of the silk solution to reach above 6%, the silk solution was dialyzed against a ˜10% (w/v) PEG solution in accordance with a method known in the art (See, e.g., Ref. 23). Purified silk fibroin solution was stored at ˜4° C.

Silk-PEG biomaterial preparation: Synthesis of a silk-PEG biomaterial was performed in accordance with a method described in Serban et al. J Biomed Mater Res A 2011; 98: 567-575. For example, a three-part biomaterial was formulated, which consisted of purified silk protein solution (e.g., 10% w/v) blended with a two-part polyethylene glycol (PEG) gelation system. In some embodiments, the chemically active PEG gelation system consisted of 4-arm PEG maleimide (˜10 kDa; Nanocs New York, N.Y.) and 4-arm PEG thiol (˜10 kDa; Creative PEGWorks, Winston-Salem, N.C.). As shown in FIG. 1, solution A was PEG maleimide (10% w/v) dispersed in silk fibroin solution, while solution B was PEG thiol (10% w/v) dispersed in silk fibroin solution. The concentration of the silk fibroin solution varied from about 6% to about 15%. Solutions were vortexed at room temperature to promote further mixing. For controls, PEG maleimide and PEG thiol were dissolved in distilled water (˜10% w/v) with no silk fibroin protein.

Gel formation and injection into tissue: These two solutions A and B were individually loaded into a double barrel syringe (e.g., 4B19, RDS/Acuflow, Holliston, Mass.) fitted with a mixing tip (2×8 blunt mixer, RDS/Acuflow) (FIG. 1). A 20 gauge needle was attached to the mixing tip. Without wishing to be bound by theory, gelation occurred in two steps (21). Initial gelation occurred within seconds after mixing as covalent cross-links formed between the maleimide and thiol functional groups. A physical crosslinking step occurs subsequently over a longer period of time and involves beta sheet formation in the silk protein chains (FIG. 1). To accelerate beta sheet formation, the cervical tissue injected with the silk-PEG material was subjected to a post-treatment that can accelerate beta-sheet formation in the silk fibroin, e.g., placing the cervical tissue in absolute alcohol such as methanol or ethanol for about 10 minutes (FIG. 1) and then stored in PBS for experiments.

Gross Morphology and Histology: Silk-PEG biomaterial (silk fibroin protein ˜7%) was injected into a cervical tissue. Gross images of the tissue before and after injection were obtained. The tissue was then formalin fixed and paraffin embedded. Hematoxylin and eosin (H&E) staining was performed using standard protocols known in the art.

The injected tissue was also evaluated for mechanical properties and swelling behaviors using standardized and validated methods known to a skilled artisan as described below. The measurements were performed in triplicate. Controls included uninjected tissues and tissues injected with PEG only. Cytocompatibility was tested with primary human cervical fibroblasts.

Swelling properties: Swelling properties were assessed because PEG-based biomaterials are generally known to swell in vivo (25). To assess swelling properties, a cervical tissue was cut into cylinders (8 mm diameter) using a punch biopsy (33-37, Miltex) (FIG. 6A). Solutions of PEG-thiol and PEG-malemide were made with 10% and 15% silk as described above. Cervical tissue specimens were injected with PEG only, PEG in 10% (w/v) silk, and PEG in 15% (w/v) silk. To determine the amount of silk-PEG biomaterial that was injected, the specimens were weighed before and after injection. The specimens were placed in alcohol for about 10 min followed by 1X PBS, pH 7.4 for 15 minutes. Excess fluid was blotted and the initial weight was recorded. The tissue-silk-PEG samples were stored in 12 well plates, covered with 1X PBS, pH 7.4, incubated at 37° C. The samples were weighed at intervals of 10 minutes, 20 minutes, 40 minutes, 1 hour, 2 hours, 8 hours, 24 hours, and 48 hours. Swelling was calculated at each time interval according to the following formula: The percent increase in sample weight, W_{increase}, was calculated as:

W_{increase}=100(1+(wₜ−wₒ)/wₒ) (Eq.1)

where wₒ is the initial weight and wₜ is the weight measured after a given amount of time.

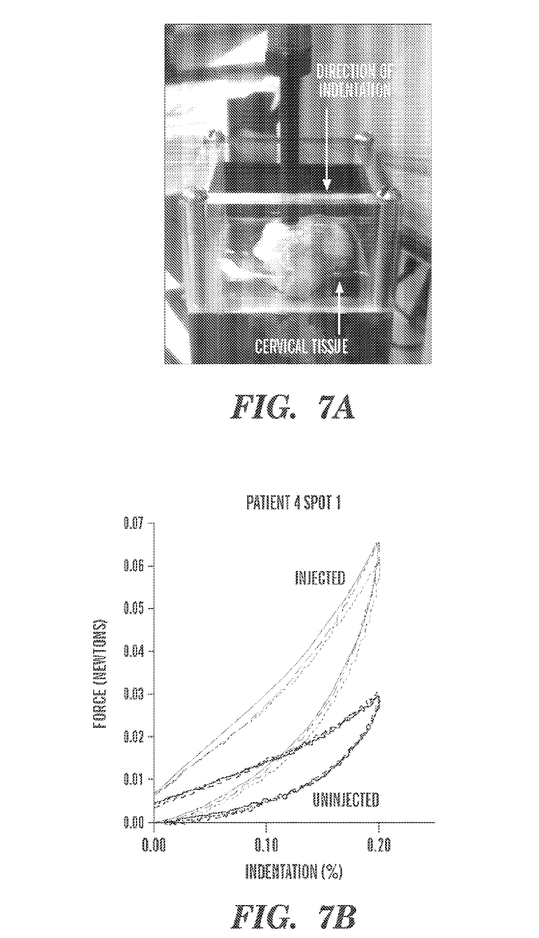

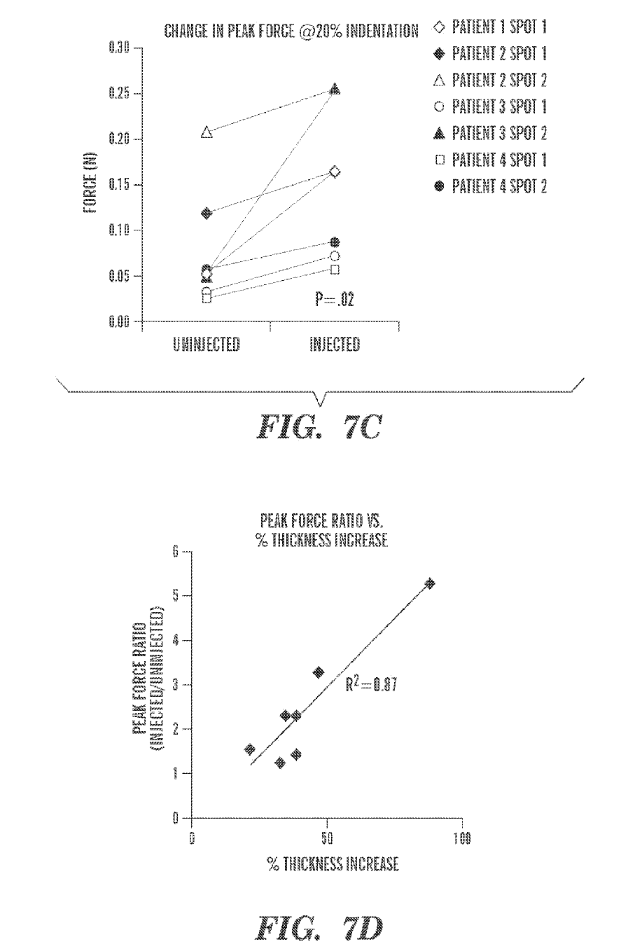

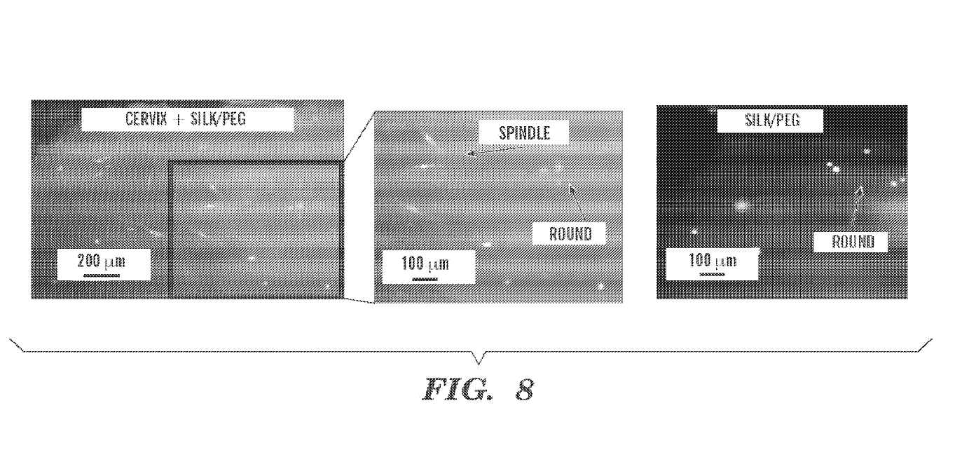

Mechanical testing: Cervical tissue specimens were thawed in PBS at room temperature. Mechanical testing was performed in a custom designed acrylic fixture filled with PBS (FIG. 5A and FIG. 7A). Testing was performed by tissue indentation with a solid rod. A universal material testing machine (Zwick Z2.5/TS1S, Ulm, Germany) with a ˜20 N load cell was used to collect indentation data. The machine controlled the displacement of the tissue according to user-defined testing protocols. Optimal loading protocols were previously determined. Each cervical specimen was subjected to ˜20% indentation at ˜0.1 mm/sec and a load-displacement curve was recorded. This cyclic loading protocol was repeated 3 times at each test site to assess repeatability. The test site was marked and the tissue was injected with ˜0.5-˜1.0 mL of silk-PEG biomaterial (silk fibroin ˜7% w/v). The treated specimens were placed in alcohol for 10 min followed by 1X PBS for 15 min. Repeat indentation testing was then performed at the same site. Peak force and change in the thickness of the tissue was measured.

Cytocompatibility assay: Human cervical fibroblasts were cultured from a cervical tissue using an explant technique as known in the art (See, e.g., Ref. 26). For example, minced cervical tissue was cultured in DMEM, 10% fetal bovine serum, and antibiotics. After 10 days, cervical fibroblasts grew out from the explants. The fibroblasts were culture expanded and cryopreserved using standard techniques.

The fibroblasts were cultured on two surfaces: silk-PEG only (control) and cervical tissue injected with silk-PEG. For cervical tissue, a cervical biopsy (3 cm×3 cm×0.3 cm) was harvested and used fresh. Silk-PEG (silk fibroin protein ˜7% w/v) was injected into cervical tissue. The tissue-silk-PEG was divided along the long axis to expose a surface with tissue and silk-PEG. The exposed surface was seeded with ˜100,000 fibroblasts and placed in culture media. As a control, the silk-PEG biomaterial (no tissue) was seeded with fibroblasts. The seeded tissue and control were incubated at 37° C. for 48 h. Cell viability was assessed using the Live/Dead viability/cytotoxicity kit (Molecular Probes, Life Technologies, NY) by incubation with 2 μM ethidium and 1 μM calcein AM at room temperature for about 30 min. Uptake of calcein AM occurs in live cells and is subsequently converted to calcein, giving the cells a green fluorescence. Ethidium enters damaged cells and produces a red fluorescence by binding to nucleic acid. Cell morphology was visualized using inverted fluorescence microscopy (Axiovert S100, Leica).

Statistical analysis: Student's t-test was used to analyze continuous variables and a paired t-test was used to analyze paired samples. Analysis of tissue swelling was assessed with two-way ANOVA (GraphPad Prism ver. 5.04, GraphPad Software, San Diego Calif.). A p-value <0.05 was considered statistically significant.

### Experimental Results

Cervical tissue specimens: A total of 8 patients were consented for removal of cervical tissue specimens post-hysterectomy (Caucasian: N=4, African American: N=3, Hispanic: N=1). The age of patients ranged between 37-54 years (median=47 years). The indications for hysterectomy were fibroid uterus/menorrhagia (N=6), dysfunctional uterine bleeding (N=1), and prophylactic due to a history of Lynch syndrome (N=1).

Gel formation and tissue injection: Rapid gelation (seconds) of a translucent, white material was observed after solution A and solution B were injected through the mixing tip and 20 gauge needle. For example, initial gelation of the silk fibroin/PEG biomaterial occurred about 5 seconds after injection due to cross-link formations between thiol and maleimide functional groups (FIG. 1: Phase 1). Further stiffening occurred with silk beta sheet formation, e.g., further exposing the silk fibroin/PEG biomaterial to alcohol treatment such as methanol or ethanol (FIG. 1; Phase 2). Gelation was confirmed by the vial inversion test as known in the art (see, e.g., Ref. 21) (FIG. 1). After about 10 seconds of injection, gelation of the biomaterial within the mixing tip prevented further injection and a new mixing tip was necessary. FIG. 2B shows changes in the shape and size of cervical tissue after injection. Analyses of specimens using H&E staining showed that the silk-PEG biomaterial was incorporated into the cervical tissue specimen after injection (FIG. 2C).

Swelling properties: Upon injection of the silk fibroin/PEG biomaterial into the cervical tissue, the mean ±SD increase in tissue wet weight was 17.0±6.7% (FIG. 3). As shown in FIG. 6B, swelling properties of the tissue injected with a mixture of PEG and silk fibroin (10% and 15% w/v silk) were not significantly different in phosphate buffered saline (PBS) than native tissue controls. However, tissue swelling was significantly increased when injected with PEG only (p<0.01).

Mechanical testing 1: Subjected to the uniaxial compression (FIG. 5A), the peak stress at 25% compressive strain was doubled in the injected tissue compared to uninjected controls (p<0.001)(FIG. 5C).

Mechanical testing 2: Indentation testing was performed on cervical tissue from 4 subjects before and after injection of silk-PEG biomaterial (˜7% w/v silk fibroin) at predetermined sites on the tissue (FIG. 7A). The load-unload response was consistent between testing cycles (FIG. 7B) indicating the loading protocol did not damage the tissue. The maximum force needed to indent the tissue by 20% more than doubled after injection of the cervical tissue with silk-PEG biomaterial (FIG. 7C). The mean ±S.D. ratio of maximum force for to injected/uninjected tissue was 2.5±1.4, (p=0.02).

To assess a dose-response effect, the maximum force was plotted as a function of injected biomaterial (FIG. 7D). The amount of injected biomaterial was quantified by measuring the thickness of the tissue before and after injection and calculating the percent thickness increase (0% corresponds to no change in thickness; 100% corresponds to doubled thickness). FIG. 7D shows a dose response effect. As more biomaterial was injected, the peak force significantly increased and varied linearly with the increase in thickness (R2=0.87, p=0.002).

Cytocompatibility assay: Cervical cells, e.g., cervical fibroblasts, remained viable for at least 48 hours when cultured on the silk fibroin/PEG biomaterial. As shown in FIG. 8, few nonviable fibroblasts (red fluorescence) were seen on tissue-silk-PEG and silk-PEG biomaterial. Viable fibroblasts (green fluorescence) were visualized on both tissue-silk-PEG and silk-PEG biomaterial. However, the morphology of the viable fibroblasts appeared different. Fibroblasts on silk-PEG biomaterial appeared rounded, probably because there were fewer cell attachment sites (21). In contrast, cells on tissue-silk-PEG displayed both rounded and spindle shaped morphology.

### Discussion

An injectable silk-PEG biomaterial that increased the stiffness of human cervical tissue (e.g., tested in vitro) was synthesized herein. The silk-PEG biomaterial gelled rapidly within the tissue and was not cytotoxic to primary cervical fibroblasts. Swelling of tissue-silk-PEG was not different from cervical tissue controls. These results indicate that an injectable silk fibroin-based composition as described herein (e.g., silk-PEG) can serve as an alternative to cervical cerclage, e.g., for treatment of cervical insufficiency.

An effective alternative to cerclage therapy can have an important impact on clinical practice. Cervical cerclage is indicated in three clinical settings: i) a known history of cervical insufficiency, ii) a prior history of preterm birth with a short cervical length (12) and iii) a dilated cervix in the midtrimester (27, 28). Although placement of a cerclage is more effective than expectant management in these settings, miscarriage or preterm birth can still occur even though cerclage is present. Lack of efficacy is seen in the following settings. First, cerclage displacement or cervical laceration (29) can occur which leads to reduced efficacy. Second, cerclage is ineffective in the setting of multiple gestations (30). Third, intrauterine infection or utero-placental bleeding can occur even though a cerclage is present. Last, cervical dilation often begins near the internal os of the cervix, but cerclage placement at this location can be technically challenging from a vaginal approach. An effective, injectable therapy that addresses one or more of these limitations of cerclage would be an exciting clinical development. In some embodiments, the methods described herein can be used to address one or more of these limitations of cerclage, and thus can be used in place of cerclage, or in concurrent with a cerclage treatment.

The cervical extracellular matrix (ECM) is composed of fibrous connective tissue. The most important constituent of the ECM is fibrillar collagen (11). Key ECM molecules that affect the collagen network include hyaluronan (31), proteoglycans (32) and water (33). Previous report has shown the macroscopic mechanical properties of cervical tissue arisen from this fibrous ECM (11, 34).

A cervical cerclage does not change the properties of cervical tissue. In contrast, various embodiments presented herein describes methods that can be used, e.g., to improve the properties of a cervical tissue (e.g., mechanical stiffness of a cervical tissue) by integration of a silk fibroin-based composition or gel into the cervical tissue, which can in turn provide better support than a cerclage. In addition, an injectable silk fibroin-based composition can be easier to place than a cerclage because a straight needle has improved access to the upper part of the cervix compared with a curved needle used for cervical cerclage placement.

In one embodiment, silk and PEG were selected for use in a silk fibroin-based composition in the method described herein because both materials are biodegradable, biocompatible, and approved by the Food and Drug Administration (FDA) for use in the body. Silk sutures are classified as non-absorbable because they retain tensile strength at least 60 days in vivo (35). The rate of degradation of silk biomaterials can by be accelerated or delayed by changing the method of purification, silk morphology and/or silk concentration (36). In addition, the biocompatibility of silk films in rats was found to be comparable to other commonly used biomaterials (37). COSEAL™ is a PEG-based surgical sealant approved to achieve hemostasis in the setting of vascular reconstructions. Combining silk with PEG can enhance the mechanical properties and decrease the swelling of PEG alone (21).

In some embodiments, an injectable silk fibroin-based composition for cervical support is desirable to have a well-defined degradation profile, e.g., a time-dependent degradation profile to meet the requirements of pregnancy. For example, in the midtrimester and early third trimester, a silk fibroin-based composition, upon injection, should stiffen the cervical tissue to prevent cervical shortening. Close to term, the silk fibroin-based composition should be degraded to allow normal cervical dilation. Complete degradation of silk scaffolds in rats varying from 2 months to 1 year has been previously reported to depend on the purification process (36). The silk protein concentration also affected the degradation profile (36). Accordingly, a degradation profile of a silk fibroin-based composition can be adjusted for requirements of pregnancy, e.g., by varying silk purification process, silk processing method, and/or concentration of silk fibroin.

Non-pregnant cervical tissue is significantly stiffer than cervical tissue from pregnancy (33). While human cervical tissue used in this Example was obtained from non-pregnant women, the silk-PEG biomaterial still demonstrated its capability to stiffen non-pregnant human cervical tissue, indicating the stiffening effect of the silk-PEG biomaterial in a cervical tissue from pregnancy can be more significant. In addition, the silk-PEG biomaterial was not cytotoxic to primary human cervical fibroblasts, which are the most plentiful cells in the cervical stroma.

In some embodiments, it is desirable to accelerate beta-sheet formation in the silk-PEG biomaterial upon injection, e.g., when silk-PEG in the absence of beta-sheet formation is not sufficient to demonstrate desirable mechanical properties of a cervical tissue. As shown in the Example, acceleration of beta-sheet formation in a silk-PEG biomaterial can be performed with exogenous ethanol. Alternate methods of inducing beta sheet formation in silk are known in the art and can include, but are not limited to, sonication, physical agitation, electrogelation, and any combinations thereof (16-18).

Presented herein is an example of a silk fibroin-based, biocompatible, and injectable biomaterial for treatment of cervical insufficiency. In some embodiments, cervical tissues injected with silk fibroin-based biomaterial were twice as stiff as the uninjected controls. In other embodiments, the silk fibroin-based biomaterial is injected into a cervix of an animal (e.g., mice, porcine, canine, rabbits) to increase the mechanical stiffness of the cervix.

### REFERENCES

1. Martin J A, Hamilton B E, Sutton P D, et al. Births: final data for 2007. Natl Vital Stat Rep 2010:58;1-85.
2. Institute of Medicine: Preterm Birth: Causes, Consequences, and Prevention. Washington D.C.: National Academies Press, 2006.
3. Mathews T J, MacDorman M F. Infant mortality statistics from the 2006 period linked birth/infant death data set. Natl Vital Stat Rep 2010; 58:1-31.
4. Saigal S, Doyle L W. An overview of mortality and sequelae of preterm birth from infancy to adulthood. Lancet 2008; 371:261-269.
5. Iams J D, Goldenberg R L, Meis P J, et al. The length of the cervix and the risk of spontaneous premature delivery. N Engl J Med 1996; 334:567-572.
6. Owen J, Yost N, Berghella V, et al. Mid-trimester endovaginal sonography in women at high risk for spontaneous preterm birth. JAMA 2001; 286:1340-1348.
7. Fonseca E B, Celik E, Parra M, et al. Progesterone and the risk of preterm birth among women with a short cervix. N Engl J Med 2007; 357:462-469.
8. Hassan S S, Romero R, Vidyadhari D, et al. Vaginal progesterone reduces the rate of preterm birth in women with a sonographic short cervix: a multicenter, randomized, double-blind, placebo-controlled trial. Ultrasound Obstet Gynecol 2011; 38:18-31.
9. Iams J D, Berghella V. Care for women with prior preterm birth. Am J Obstet Gynecol 2010; 203:89-100.
10. House M, Socrate S. The cervix as a biomechanical structure. Ultrasound Obstet Gynecol 2006; 28:745-749.
11. House M, Kaplan D L, Socrate S. Relationships between Mechanical Properties and Extracellular Matrix Constituents of the Cervical Stroma during Pregnancy. Semin Perinatol 2009; 33:300-307.
12. Owen J, Hankins G, Iams J D, et al. Multicenter randomized trial of cerclage for preterm birth prevention in high-risk women with shortened midtrimester cervical length. Am J Obstet Gynecol 2009; 201:e371-378.
13. Omenetto F G, Kaplan D L. New opportunities for an ancient material. Science 2010; 329:528-531.
14. Hu X, Kaplan D, Cebe P. Determining beta-sheet crystallinity in fibrous proteins by thermal analysis and infrared spectroscopy. Macromolecules 2006; 39:6161-6170.
15. Hu X, Kaplan D, Cebe P. Dynamic protein-water relationships during beta-sheet formation. Macromolecules 2008; 41:3939-3948.
16. Wang X, Kluge J A, Leisk G G, Kaplan D L. Sonication-induced gelation of silk fibroin for cell encapsulation. Biomaterials 2008; 29:1054-1064.
17. Yucel T, Cebe P, Kaplan D L. Vortex-induced injectable silk fibroin hydrogels. Biophys J 2009; 97:2044-2050.
18. Yucel T, Kojic N, Leisk G G, Lo T J, Kaplan D L. Non-equilibrium silk fibroin adhesives. J Struct Biol 2010;170:406-412.
19. Hu X, Wang X, Rnjak J, Weiss A S, Kaplan D L. Biomaterials derived from silk-tropoelastin protein systems. Biomaterials 2011; 31:8121-8131.
20. Hu X, Lu Q, Sun L, et al. Biomaterials from Ultrasonication-Induced Silk Fibroin-Hyaluronic Acid Hydrogels. Biomacromolecules 2010.
21. Serban M A, Panilaitis B, Kaplan D L. Silk fibroin and polyethylene glycol-based biocompatible tissue adhesives. J Biomed Mater Res A 2011; 98:567-575.
22. Kim U J, Park J, Kim H J, Wada M, Kaplan D L. Three-dimensional aqueous-derived biomaterial scaffolds from silk fibroin. Biomaterials 2005; 26:2775-2785.
23. Lovett M, Cannizzaro C, Daheron L, Messmer B, Vunjak-Novakovic G, Kaplan D L. Silk fibroin microtubes for blood vessel engineering. Biomaterials 2007; 28:5271-5279.
24. Chen X, Shao Z, Marinkovic N S, Miller L M, Zhou P, Chance M R. Conformation transition kinetics of regenerated Bombyx mori silk fibroin membrane monitored by time-resolved FTIR spectroscopy. Biophys Chem 2001; 89:25-34.
25. Spotnitz W D, Burks S. Hemostats, sealants, and adhesives: components of the surgical toolbox. Transfusion 2008; 48:1502-1516.
26. House M, Sanchez C C, Rice W L, Socrate S, Kaplan D L. Cervical tissue engineering using silk scaffolds and human cervical cells. Tissue Eng Part A 2010; 16:2101-2112.
27. Pereira L, Cotter A, Gomez R, et al. Expectant management compared with physical examination-indicated cerclage (EM-PEC) in selected women with a dilated cervix at 14(0/7)-25(6/7) weeks: results from the EM-PEC international cohort study. Am J Obstet Gynecol 2007; 197:483 e481-488.
28. Daskalakis G, Papantoniou N, Mesogitis S, Antsaklis A. Management of cervical insufficiency and bulging fetal membranes. Obstet Gynecol 2006; 107:221-226.
29. Landy H J, Laughon S K, Bailit J L, et al. Characteristics associated with severe perineal and cervical lacerations during vaginal delivery. Obstet Gynecol 2011; 117:627-635.
30. Berghella V, Odibo A O, To M S, Rust O A, Althuisius S M. Cerclage for short cervix on ultrasonography: meta-analysis of trials using individual patient-level data. Obstet Gynecol 2005; 106:181-189.
31. Straach K J, Shelton J M, Richardson J A, Hascall V C, Mahendroo M S. Regulation of hyaluronan expression during cervical ripening. Glycobiology 2005; 15:55-65.
32. Osmers R, Rath W, Pflanz M A, Kuhn W, Stuhlsatz H W, Szeverenyi M. Glycosaminoglycans in cervical connective tissue during pregnancy and parturition. Obstet Gynecol 1993; 81:88-92.
33. Myers K M, Paskaleva A P, House M, Socrate S. Mechanical and biochemical properties of human cervical tissue. Acta Biomater 2008; 4:104-116.
34. Myers K M, Socrate S, Paskaleva A P, House M. A Study of the Anisotropy and Tension/Compression Behavior of Human Cervical Tissue. J Biomech Eng 2010; 132:021003-021015.
35. Altman G H, Diaz F, Jakuba C, et al. Silk-based biomaterials. Biomaterials 2003; 24:401-416.
36. Wang Y, Rudym D D, Walsh A, et al. In vivo degradation of three-dimensional silk fibroin scaffolds. Biomaterials 2008; 29:3415-3428.
37. Meinel L, Hofmann S, Karageorgiou V, et al. The inflammatory responses to silk films in vitro and in vivo. Biomaterials 2005; 26:147-155.

Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method for increasing mechanical stiffness of a cervical tissue in a subject, comprising placing a silk fibroin-based composition into at least a portion of a cervix of the subject, wherein the silk fibroin-based composition forms a gel upon placement into the cervix, thereby increasing the mechanical stiffness of the cervical tissue in the subject.

2. The method of claim 1, wherein the placing comprises injecting the silk fibroin-based composition into said at least a portion of the cervix of the subject.

3. The method of claim 1, wherein the at least a portion of the cervix is a stroma of the cervix.

4. The method of claim 1, wherein the silk fibroin-based composition comprises silk fibroin having a concentration of about 5 wt % to about 30 wt %.

5. The method of claim 4, wherein the silk fibroin has a concentration of about 5 wt % to about 10 wt %.

6. The method of claim 1, wherein the silk fibroin-based composition comprises at least two functionally-activated PEG components capable of reacting with one another to form a crosslinked matrix, and the silk fibroin capable of forming beta-sheets to further stabilize the crosslinked matrix.

7. The method of claim 6, further comprising mixing the PEG components and the silk fibroin.

8. The method of claim 6, wherein each PEG component is a four-armed PEG.

9. The method of claim 6, wherein one of the PEG components is functionally activated with a maleimidyl group.

10. The method of claim 6, wherein one of the PEG components is functionally activated with a thiol group.

11. (canceled)

12. The method of claim 1, further comprising exposing the silk fibroin-based composition, upon the placement into the cervix, to a treatment comprising ultrasound for a sufficient period of time to initiate gelation.

13. The method of claim 1, further comprising exposing the silk fibroin-based composition, upon the placement into the cervix, to a treatment comprising an electric field for a sufficient period of time to initiate gelation.

14. The method of claim 1, further comprising exposing the silk fibroin-based composition, upon the placement into the cervix, to an alcohol treatment or a water-annealing treatment.

15. (canceled)

16. The method of claim 1, wherein the subject is at risk of, or diagnosed with cervical insufficiency.

17. The method of claim 1, wherein the subject has a history of preterm birth.

18-21. (canceled)

22. The method of claim 1, wherein the silk fibroin-based composition further comprises an active agent.

23. The method of claim 22, wherein the active agent is selected from the group consisting of cells, proteins, peptides, nucleic acids, aptamers, antibodies or fragments or portions thereof, antigens, hormones, hormone antagonists, growth factors or recombinant growth factors and fragments and variants thereof, cell attachment mediators, enzymes, antibiotics, viruses, prodrugs, therapeutic agents, small molecules, and any combinations thereof.

24. The method of claim 23, wherein the active agent is a population of cervical cells.

25. The method of claim 23, wherein the active agent is selected from a group consisting of thrombospondin 2, thrombin, fibrin, cyanoacrylate, glutaraldehyde, and any combinations thereof.

26. The method of claim 1, wherein the silk fibroin-based composition further comprises an extracellular matrix selected from a group consisting of collagen, proteoglycans, hyaluronan, elastin, and any combinations thereof.

27-54. (canceled)
